(19) United States
(12) Patent Application Publication
(10) Pub. No.: US 2018/0119035 A1
(43) Pub. Date: May 3, 2018

(54) PRODUCTION OF BIOGAS AND/OR ETHANOL FROM WASTE MATERIAL

(71) Applicant: infimer Technologies Ltd., Moshav Avihayil (IL)

(72) Inventor: Yuval TAMIR, Moshav Avihayil (IL)

(21) Appl. No.: 15/572,529

(22) PCT Filed: May 10, 2016
(86) PCT No.: PCT/IL2016/050496

§ 371 Date: Nov. 8, 2017

Related U.S. Application Data
(60) Provisional application No. 62/159,945, filed on May 11, 2015.

(51) Int. Cl. C10L 1/02; B03B 5/30; B03B 9/06; C12P 7/10; C12P 5/02; C12M 1/107; C12M 1/00; C10L 3/08; B09B 3/00

(52) U.S. Cl. CPC C10L 1/02; B03B 5/30; B03B 9/06; C12P 7/10; C12P 5/023; C12P 2201/00; C12M 21/12; C12M 45/03; C10L 3/08; B09B 3/00; C12M 21/04

(57) ABSTRACT

A method and a system for processing waste material to form a biogas and/or ethanol are disclosed herein. The method comprises subjecting waste material to separation according to specific gravity, to thereby obtain a fraction which is a separated lignocellulose; and processing the separated lignocellulose to obtain the biogas and/or ethanol. The system comprises at least one separator configured for separating materials in waste material according to specific gravity to obtain a first fraction comprising a low density material and a second fraction comprising a high-density material; and a bioreactor or bioreactor system configured for processing the separated lignocellulose to thereby obtain the biogas and/or ethanol. The separator contains an aqueous liquid selected such that a portion of the waste material sinks and another portion does not sink upon contact with the aqueous liquid.

### FIELD AND BACKGROUND OF THE INVENTION

The present invention, in some embodiments thereof, relates to waste treatment and, more particularly, but not exclusively, to methods and systems for producing biogas and/or ethanol by processing waste material.

Biomass is an organic matter that may be used as an energy source. Biomass is typically derived from sources such as agricultural and municipal wastes. Energy production from biomass is typically performed while utilizing carbohydrates present in a waste material, such as lignocellulose, and involves anaerobic digestion mediated by microorganisms and/or enzymes. The products obtained by biomass processing depend on the microorganisms used and on the nature of the material subjected to anaerobic digestion.

Biogas is typically a mixture of different gases, which consists primarily of methane and carbon dioxide, and may include also small amounts of hydrogen sulfide, moisture and/or siloxanes. Biogas is produced by the breakdown of biomass in the absence of oxygen, by anaerobic digestion, typically with anaerobic bacteria, which digest material inside a closed system.

Ethanol, which is a biofuel, may be generated from carbohydrates present in waste, such as lignocellulose. In ethanol production, complex organic polymers are first broken down, typically by enzymatic hydrolysis, into monomeric, soluble sugars, and the sugars resulting from the hydrolysis are then fermented, typically in the presence of yeast, distilled and purified into useable ethanolic biofuel.

Lignocellulose includes hemicellulose, lignin, and cellulose. The long-chain cellulose and hemicelluloses molecules are resistant to degradation by microorganisms. Lignin is also resistant to degradation by microorganisms and their related enzymes, and hence energy production from biomass involves separating the cellulose and hemicellulose bound to lignin, and converting the cellulose and hemicelluloses into fermentable/digestible simple sugar solutions.

There are several basic techniques for converting lignocellulosic biomass, including cellulose, hemicellulose, and lignin, into fermentable/digestible simple sugar solutions which can be used for energy production. One exemplary technique includes a one-step acid hydrolysis in which the hemicellulose and cellulose are broken down in a single step using concentrated aqueous solutions of strong mineral acids. A second exemplary technique is a two-step dilute acid process in which the hemicellulose and cellulose parts are hydrolyzed separately. Another technique involves an enzymatic process in which the lignocellulosic biomass is first pretreated in order to increase accessibility for the cellulolytic enzymes. The enzymatic process is also a two-step hydrolysis technique although the cellulose fraction is broken down using cellulases instead of acids.

U.S. Pat. No. 6,368,500 describes a system for treatment of collected waste, the system comprising at least one separator for separating between first waste material having a specific gravity equal or less than that of water and second waste material having a specific gravity above that of water; at least one crusher for producing a liquid product from the first waste material; and acetogenic and methanogenic fermentors for fermenting the liquid product.

Additional background art includes International Patent Applications having Publication Nos. WO 2005/077630, WO 2005/092708, WO 2006/035441, WO 2006/079842 and WO 2010/082202; European Patent No. 1711323; KR 2003/0014929; U.S. Pat. Nos. 3,850,771, 4,013,616, 4,772,430, 4,968,463, 5,217,655, 6,017,475, 6,253,527, 6,423,254, and 7,497,335; and U.S. Patent Applications having Publication Nos. 2005/0026262, 2004/0080072 and 2004/0080072.

### SUMMARY OF THE INVENTION

According to an aspect of some embodiments of the present invention there is provided a method of processing a waste material so as to form a biogas and/or ethanol, the method comprising: subjecting the waste material to a separation according to specific gravity, to thereby obtain at least one fraction which is a separated lignocellulose; and processing the separated lignocellulose, to thereby obtain the biogas and/or ethanol.

According to some of the any of the embodiments described herein, processing the separated lignocellulose is performed so as to produce biogas, the processing comprising subjecting the separated lignocelluloses to a microbial digestion in the presence of an acetogenic microorganism and a methanogenic microorganism.

According to some of the any of the embodiments described herein, processing the separated lignocellulose is performed so as to produce ethanol, the processing comprising subjecting the separated lignocelluloses to a fermentation in the presence of a fermenting organism.

According to some of the any of the embodiments described herein, the method further comprises, prior to or concomitant with the processing, pre-treating the separated lignocellulose so as to at least partially decompose the lignocellulose into lignin, hemicelluloses and cellulose.

According to some of the any of the embodiments described herein, the separation according to specific gravity comprises contacting the waste material with an aqueous liquid selected such that a portion of the waste material sinks and another portion does not sink, thereby separating waste material into a first fraction comprising a low density material and a second fraction comprising a high-density material.

According to some of the any of the embodiments described herein, the first fraction comprises the separated lignocellulose.

According to some of the any of the embodiments described herein, the separation process comprises contacting the waste material with a first aqueous liquid selected such that a portion of the waste material sinks, thereby obtaining the second fraction comprising the high-density material and the first fraction comprising the low-density material, and further contacting at least one of the first fraction and the second fraction with a second aqueous liquid selected such that a portion of the fraction sinks, thereby obtaining a third fraction comprising a low-density material which does not sink in either of the aqueous liquids, a fourth fraction comprising an intermediate-density material which sinks in one of the aqueous liquids, and a fifth fraction comprising a high-density material which sinks in both of the aqueous liquids.

According to some of the any of the embodiments described herein, a specific gravity of one of the first aqueous liquid and the second aqueous liquid is at least 1.05, and a specific gravity of the other of the first aqueous liquid and the second aqueous liquid is no more than 1.01.

According to some of the any of the embodiments described herein, the intermediate-density material comprises the separated lignocellulose.

According to an aspect of some embodiments of the present invention there is provided a system for processing a waste material so as to form a biogas and/or ethanol, the system comprising: at least one separator configured for separating materials in the waste material according to specific gravity so as to obtain at least two fractions, the fractions comprising at least a first fraction which comprises a low density material and at least a second fraction which comprises a high-density material, the separator containing an aqueous liquid selected such that a portion of the waste material sinks and another portion does not sink upon contact with the aqueous liquid, thereby obtaining the first fraction and the second fraction; and a bioreactor or a bioreactor system configured for processing the separated lignocellulose to thereby obtain the biogas and/or ethanol.

According to some of the any of the embodiments described herein, the bioreactor or a bioreactor system is configured for processing the separated lignocellulose so as to produce the biogas, the processing comprising subjecting the separated lignocellulose to a microbial digestion in the presence of an acetogenic microorganism and a methanogenic microorganism.

According to some of the any of the embodiments described herein, the bioreactor or a bioreactor system is configured for processing the separated lignocellulose so as to produce ethanol, the processing comprising subjecting the separated lignocelluloses to a fermentation in the presence of a fermenting organism.

According to some of the any of the embodiments described herein, the bioreactor or bioreactor system is in communication with (is operably linked to) at least one of the at least one separator, and is configured for processing at least a portion of the first fraction which comprises a low-density material.

According to some of the any of the embodiments described herein, the at least one separator comprises a first separator containing a first aqueous liquid and a second separator containing a second aqueous liquid, the first separator and the second separator being in communication (being operably linked), and the second separator being configured for receiving at least one fraction from the first separator, and for separating the fraction received from the first separator according to specific gravity, the second aqueous liquid being selected such that a portion of the fraction received from the first separator sinks, thereby obtaining a third fraction comprising a low-density material which does not sink in either of the aqueous liquids, a fourth fraction comprising an intermediate-density material which sinks in one of the aqueous liquids, and a fifth fraction comprising a low-density material which sinks in both of the aqueous liquids.

According to some of the any of the embodiments described herein, a specific gravity of one of the first aqueous liquid and the second aqueous liquid is at least 1.05, and a specific gravity of the other of the first aqueous liquid and the second aqueous liquid is no more than 1.01.

According to some of the any of the embodiments described herein, the second separator is configured for obtaining a separated lignocellulose, the intermediate-density material comprising the lignocellulose.

According to some of the any of the embodiments described herein, the bioreactor or bioreactor system is in communication with the second separator, sand is configured for processing at least a portion of the fourth fraction which comprises an intermediate-density material.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart depicting a method of producing biogas and/or ethanol from waste material according to some embodiments of the invention;

FIG. 2 is a schematic illustration of a system for producing biogas and/or ethanol from waste material according to some embodiments of the invention; and

FIGS. 3A-B present NMR spectra of a filtrate of sea salt aqueous solution (about 20 weight percents) (FIG. 3A) and fresh water (FIG. 3B), each filtrate being obtained after 3 hours incubation with plant biomass.

### DESCRIPTION OF SPECIFIC EMBODIMENTS OF THE INVENTION

The present invention, in some embodiments thereof, relates to waste treatment and, more particularly, but not exclusively, to methods and systems for producing a biogas and/or ethanol by processing waste material.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present inventor has uncovered that contacting waste materials (e.g., unsorted waste materials) with an aqueous liquid can be utilized to advantageously separate lignocellulose from the waste material, and that the separated lignocelluloses can be efficiently utilized for production of biogas and biofuel.

As discussed hereinabove, one of the problems associated with efficient production of biogas and/or ethanol from lignocelluloses present in waste materials is the non-degradability of the cellulose, hemicelluloses and lignin molecules composing the lignocellulose, which require pre-treatment of the lignocelluloses to first dissociate the cellulose and hemicelluloses from lignin, and then hydrolyse the cellulose and hemicelluloses into shorter, simpler carbohydrates.

The present inventor has uncovered that contacting waste materials (e.g., unsorted waste materials) with an aqueous liquid can be further utilized for breaking down the lignocellulose into components which are more readily fermentable/digestible in processes for biogas and/or ethanol production, such as carbohydrates and even glucose. The present inventor has therefore devised a method and system for efficiently producing biogas and/or ethanol from waste materials.

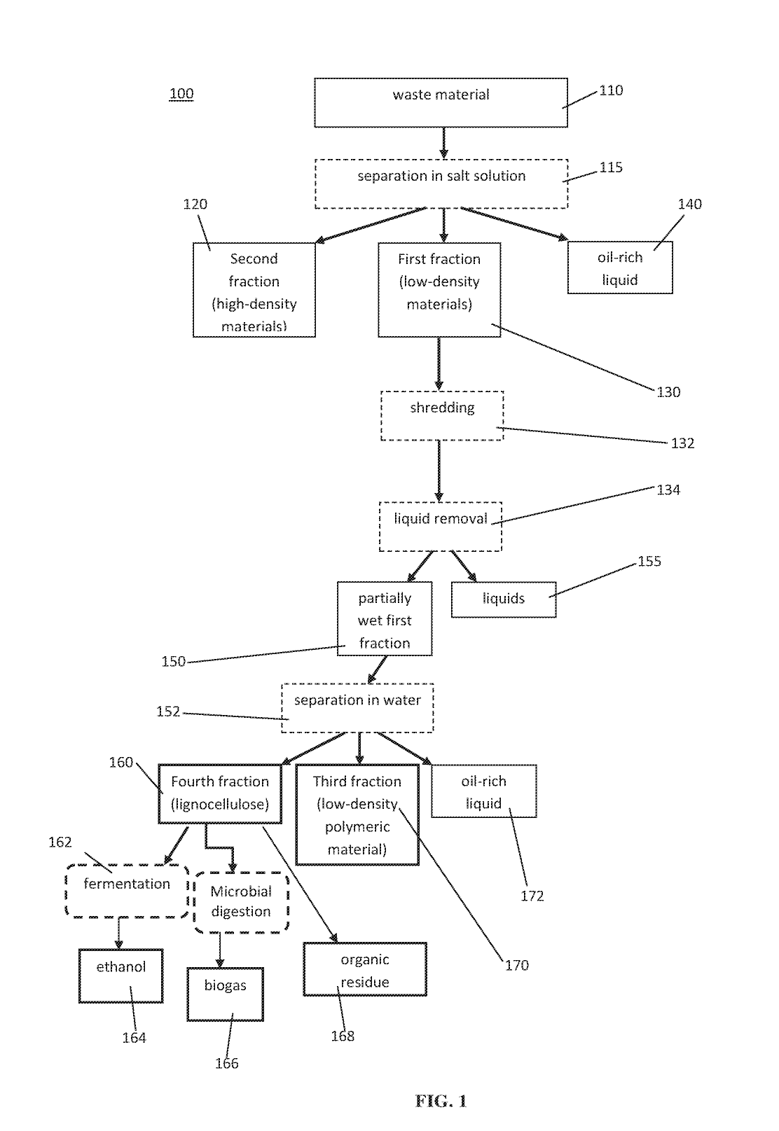

Referring now to the drawings, FIG. 1 illustrates a general procedure for producing biogas and/or ethanol from waste material utilizing contact of waste material with an aqueous solution, according to exemplary embodiments of the invention.

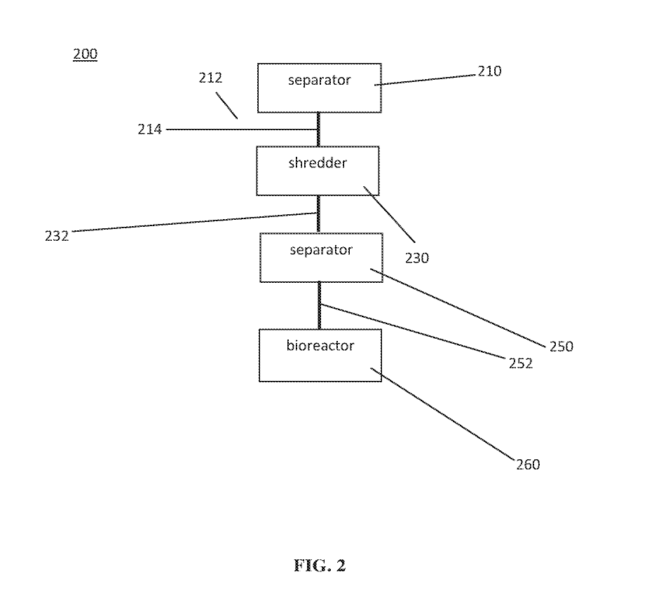

FIG. 2 is a schematic illustration of a system which can be utilized for the production of biogas and/or ethanol from waste material. The system and method presented in FIGS. 1 and 2 are described in further detail hereinbelow and in the Examples section that follows.

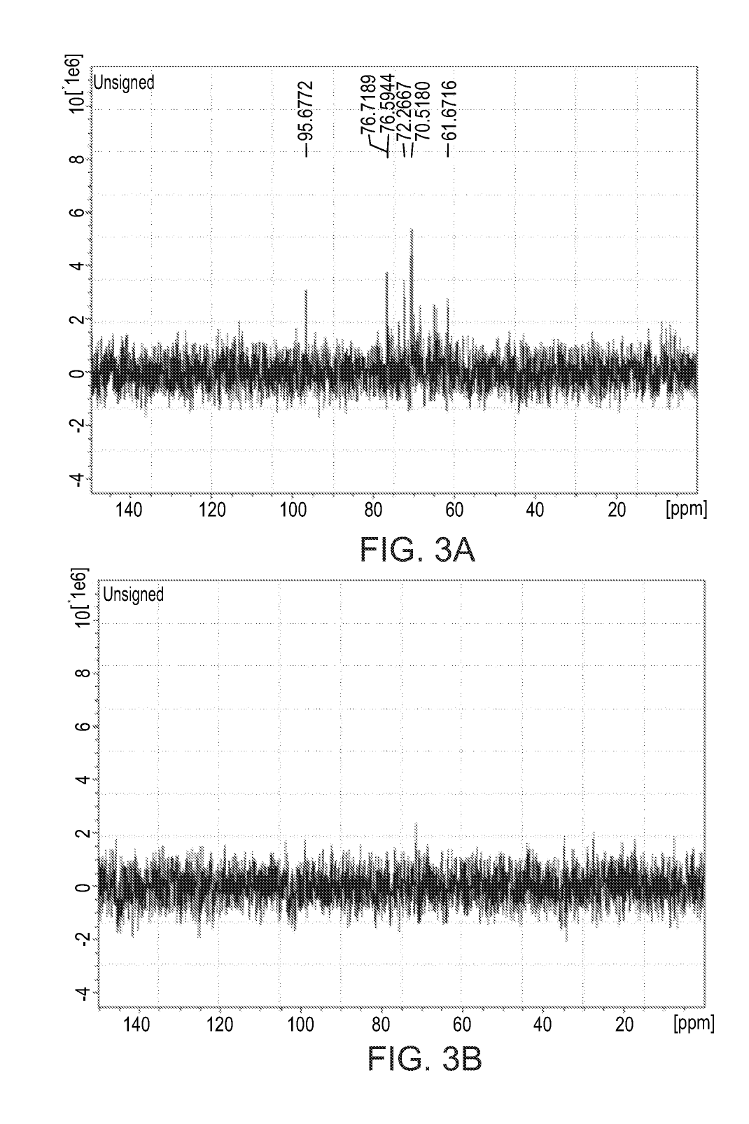

FIGS. 3A-B presents NMR spectra showing that hypertonic solution facilitates release of carbohydrates from biomass.

According to an aspect of some embodiments of the present invention, there is provided a method of processing waste material so as to produce a biogas and/or ethanol (e.g., bioethanol). A lignocellulose-enriched fraction is separated from the waste material, and the separated lignocellulose is processed to produce biogas and/or ethanol. In some embodiments, the method according to this aspect of the present invention is effected by subjecting the waste material to a separation according to specific gravity, to thereby obtain at least one fraction which is a lignocelluloses-enriched fraction or which comprises a separated lignocellulose as defined herein. In some embodiments, the separated lignocellulose is subjected to a microbial digestion as described herein in any of the respective embodiments, to thereby produce biogas, as defined herein. Alternatively or additionally, the separated lignocellulose is subjected to fermentation as described herein in any of the respective embodiments, to thereby produce ethanol, or any other alcohol.

In some embodiments, the method according to this aspect of the present invention is effected by subjecting the waste material to a separation process according to specific gravity, so as to obtain at least two fractions. In some embodiments, at least one of the fractions, herein referred to as “a first fraction”, comprises one or more low-density materials, and at least one of the fractions, herein referred to as “a second fraction” comprises one or more high-density materials. Herein, low-density materials indicate lower specific gravity values than high-density materials, the term “density” being used instead of “specific gravity” merely for brevity and to enhance readability.

The waste material may optionally be any waste material comprising lignocellulose, for example, in concentrations sufficient for obtaining a separated lignocellulose according to any of the respective embodiments described herein.

In some of any of the embodiments described herein, at least 10 weight percents of the dry weight of the waste material is lignocellulose. In some embodiments, at least 20 weight percents of the dry weight of the waste material is lignocellulose. In some embodiments, at least 30 weight percents of the dry weight of the waste material is lignocellulose. In some embodiments, at least 40 weight percents of the dry weight of the waste material is lignocellulose. In some embodiments, at least 50 weight percents of the dry weight of the waste material is lignocellulose. In some embodiments, at least 60 weight percents of the dry weight of the waste material is lignocellulose. In some embodiments, at least 70 weight percents of the dry weight of the waste material is lignocellulose.

The waste material may optionally be in the form it is received at a solid waste management facility or at a waste dump or from a landfill (referred to as “unsorted” waste material), or alternatively, waste material which has undergone preliminary sorting or separation, that is, waste material (e.g., from the aforementioned sources) from which one or more components (e.g., magnetic materials) are selectively removed (partially or entirely) before being separated according to the method described herein. The waste material may include some waste from sources other than domestic waste (e.g., in combination with domestic waste), such as sludge (e.g., sewage sludge), industrial waste (e.g., discarded packaging material, discarded material from food processing and/or paper recycling) and/or agricultural waste.

The waste material typically comprises some liquid (e.g., water, oils), for example, liquids absorbed by the waste material and/or within containers, plant material and/or animal material in the waste material. It is to be appreciated that the method of separating described herein is optionally effected by contact with a liquid, so that the waste material can therefore optionally be separated without any need for prior drying of the waste material.

Herein throughout, the phrase “waste material” refers to substantially solid waste, such as municipal solid waste, which, in some embodiments, is obtained mostly from domestic sources (household waste), and is also referred to as “trash” or “garbage”. The phrase “waste material” as used herein encompasses substantially unsorted waste material (e.g., prior to removal of a portion of the materials as described herein), that is, it comprises a wide variety of substances typical of domestic waste, and optionally further encompasses waste material, as defined herein, which has undergone some separation (e.g., removal of readily recyclable items).

Herein, “animal material” refers to material which originates from an animal, and “plant material” refers to material which originates from a plant or fungus. It is noted that coal and petroleum products and the like, which originate from organisms which lived only in the distant past, are not considered herein as animal or plant material.

Some or all of the obtained separated materials according to any of the embodiments described herein may have commercial value (e.g., as a commodity),

Additionally or alternatively, the method further comprises processing one or more of the obtained separated materials (according to any of the embodiments described herein), to thereby obtain a processed material, for example, a processed material with a commercial value that the separated material from which it is derived does not have.

Herein throughout, the term “processing” and grammatical derivations thereof, in the context of an act performed on a material (e.g., a separated material), is used to describe alteration of the composition, chemical properties and/or physical properties of the material, to thereby obtain a different, second material, referred to herein as “processed material”, having a different composition, chemical properties and/or physical properties than the material subjected to processing.

For the sake of clarity, the terms “processing” and “processed material” are used herein to describe a material obtained by procedures which include (but are not necessarily limited to) procedures other than separating, for example, by subjecting a separated material (as defined herein) to a fermentation and/or microbial digestion process as described herein.

The Separated Lignocellulose:

Herein, the phrase “separated lignocellulose” refers to an obtained material consisting primarily of lignocellulose. Thus, separated lignocellulose may comprise impurities (material other than lignocellulose), provided that at least 50 weight percents (by dry weight) of the separated material is lignocellulose (as defined herein) per se.

Preferably, the weight percentage (by dry weight) of material other than lignocellulose in the separated lignocellulose is lower than the weight percentage (by dry weight) of material other than lignocellulose in the waste material from which the separated lignocellulose is obtained. In some embodiments of any of the embodiments described herein, the weight percentage of material other than lignocellulose in the separated lignocellulose is no more than 50% of the weight percentage of material other than lignocellulose in the waste material. In some embodiments, the weight percentage of material other than lignocellulose in the separated lignocellulose is no more than 30% of the weight percentage of material other than lignocellulose in the waste material. In some embodiments, the weight percentage of material other than lignocellulose in the separated lignocellulose is no more than 20% of the weight percentage of material other than lignocellulose in the waste material. In some embodiments, the weight percentage of material other than lignocellulose in the separated lignocellulose is no more than 10% of the weight percentage of material other than lignocellulose in the waste material.

As used herein, the term “lignocellulose” (per se, rather than in a context of a separated lignocellulose, as defined herein) refers to dry matter derived from plants, which is composed primarily of carbohydrates (primarily cellulose and hemicelluloses) and lignin. Thus, an amount of lignocellulose described herein may be considered a total amount of dry matter derived from plants, regardless of the proportions of, e.g., carbohydrates and lignin. Lignocellulose is also referred to in the art as “ligneous cellulose”.

Without being bound by any particular theory, it is believed that the carbohydrates in lignocelluloses (e.g., cellulose and/or hemicelluloses) are particularly amenable to processing as described herein (e.g., as compared to lignin), including, without limitation, fermentation and/or microbial digestion processes (e.g., as described herein). The proportion of carbohydrates in the lignocellulose may optionally be enhanced by limiting an amount of lignin-rich material in the waste material being processed, for example, by using waste material with no more than a limited amount of wood (e.g., tree trimmings, lumberyard waste).

In some of any of the embodiments described herein, from 50 to 95 weight percents of the dry weight of a separated lignocellulose (as defined herein) is lignocellulose. In some embodiments, from 50 to 90 weight percents of the dry weight is lignocellulose. In some embodiments, from 50 to 85 weight percents of the dry weight is lignocellulose. In some embodiments, from 50 to 80 weight percents of the dry weight is lignocellulose. In some embodiments, from 50 to 75 weight percents of the dry weight is lignocellulose. In some embodiments, from 50 to 70 weight percents of the dry weight is lignocellulose. In some such embodiments, at least 40 weight percents of the lignocellulose per se is carbohydrates. In some embodiments, at least 60 weight percents of the lignocellulose per se is carbohydrates. In some embodiments, at least 80 weight percents of the lignocellulose per se is carbohydrates. In some embodiments, at least 90 weight percents of the lignocellulose per se is carbohydrates.

In some of any of the embodiments described herein, at least 60 weight percents of the dry weight of a separated lignocellulose (as defined herein) is lignocellulose. In some embodiments, from 60 to 95 weight percents of the dry weight is lignocellulose. In some embodiments, from 60 to 90 weight percents of the dry weight is lignocellulose. In some embodiments, from 60 to 85 weight percents of the dry weight is lignocellulose. In some embodiments, from 60 to 80 weight percents of the dry weight is lignocellulose. In some embodiments, at least 40 weight percents of the lignocellulose per se is carbohydrates. In some embodiments, at least 60 weight percents of the lignocellulose per se is carbohydrates. In some embodiments, at least 80 weight percents of the lignocellulose per se is carbohydrates. In some embodiments, at least 90 weight percents of the lignocellulose per se is carbohydrates.

In some of any of the embodiments described herein, at least 70 weight percents of the dry weight of a separated lignocellulose (as defined herein) is lignocellulose. In some embodiments, from 70 to 95 weight percents of the dry weight is lignocellulose. In some embodiments, from 70 to 90 weight percents of the dry weight is lignocellulose. In some embodiments, from 70 to 85 weight percents of the dry weight is lignocellulose. In some embodiments, from 75 to 85 weight percents of the dry weight is lignocellulose. In some such embodiments, at least 40 weight percents of the lignocellulose per se is carbohydrates. In some embodiments, at least 60 weight percents of the lignocellulose per se is carbohydrates. In some embodiments, at least 80 weight percents of the lignocellulose per se is carbohydrates. In some embodiments, at least 90 weight percents of the lignocellulose per se is carbohydrates.

In some of any of the embodiments described herein, at least 80 weight percents of the dry weight of a separated lignocellulose (as defined herein) is lignocellulose. In some embodiments, from 80 to 95 weight percents of the dry weight is lignocellulose. In some embodiments, from 80 to 90 weight percents of the dry weight is lignocellulose. In some embodiments, from 80 to 85 weight percents of the dry weight is lignocellulose. In some such embodiments, at least 40 weight percents of the lignocellulose per se is carbohydrates. In some embodiments, at least 60 weight percents of the lignocellulose per se is carbohydrates. In some embodiments, at least 80 weight percents of the lignocellulose per se is carbohydrates. In some embodiments, at least 90 weight percents of the lignocellulose per se is carbohydrates.

In some of any of the embodiments described herein, at least 90 weight percents of the dry weight of a separated lignocellulose (as defined herein) is lignocellulose. In some embodiments, from 90 to 95 weight percents of the dry weight is lignocellulose. In some such embodiments, at least 40 weight percents of the lignocellulose per se is carbohydrates. In some embodiments, at least 60 weight percents of the lignocellulose per se is carbohydrates. In some embodiments, at least 80 weight percents of the lignocellulose per se is carbohydrates. In some embodiments, at least 90 weight percents of the lignocellulose per se is carbohydrates.

In some of any of the embodiments described herein, at least 95 weight percents of the dry weight of a separated lignocellulose (as defined herein) is lignocellulose. In some such embodiments, at least 40 weight percents of the lignocellulose per se is carbohydrates. In some embodiments, at least 60 weight percents of the lignocellulose per se is carbohydrates. In some embodiments, at least 80 weight percents of the lignocellulose per se is carbohydrates. In some embodiments, at least 90 weight percents of the lignocellulose per se is carbohydrates.

Separation Process Utilizing Liquid:

As used herein, the term “specific gravity” refers to a ratio of density of a material to a density of pure water under the same conditions (e.g., temperature, pressure). Thus, the specific gravity of pure water is defined as 1. In some embodiments of any of the embodiments described herein, the specific gravity is a specific gravity at room temperature (e.g., 25° C.) and atmospheric pressure. However, because specific gravity is a ratio, it is less sensitive than density to changes in conditions (e.g., temperature, pressure). Hence, in some embodiments of any of the embodiments described herein, the specific gravity is a specific gravity under working conditions. For example, ambient temperature under working conditions may vary, for example, within a range of 0° C. to 50° C., and ambient pressure may vary according to altitude of the location.

In some embodiments of any of the embodiments described herein, the separation process comprises contacting the waste material with a liquid selected such that a portion of the waste material sinks in the liquid and another portion does not sink.

The liquid may be any type of liquid, including a pure liquid, a solution, and a suspension. In some embodiments of any of the embodiments described herein, the liquid is an aqueous liquid.

In embodiments utilizing a liquid, the waste material is separated into a fraction of low-density materials (referred to herein as a “first fraction”), comprising materials which do not sink; and a fraction of high-density materials (referred to herein as a “second fraction”), comprising materials which sink. At least one of the first and second fractions may be collected and optionally separated further, in order to obtain a separated lignocellulose according to any of the embodiments described herein.

Herein, the term “sink” encompasses sinking to a bottom of a liquid (e.g., sedimenting), as well as sinking below a surface of the liquid.

In some of any of the embodiments described herein, to “sink” refers to sinking to a bottom of a liquid (e.g., sedimenting), such that materials which sink below a surface of the liquid but do not sink to a bottom of the liquid are considered as materials which do not sink, and are optionally included in a fraction of low-density materials (e.g., a first fraction) according to any of the respective embodiments described.

In some of any of the embodiments described herein, to “sink” refers to sinking below a surface of a liquid, such that materials which sink below a surface of the liquid but do not sink to a bottom of the liquid are considered as materials sink, and are optionally included in a fraction of high-density materials (e.g., a second fraction) according to any of the respective embodiments described.

In some of any of the embodiments described herein, materials which sink below a surface of the liquid but do not sink to a bottom of the liquid are not included in either a fraction of low-density materials (e.g., a first fraction) or a fraction of high-density materials (e.g., a second fraction) according to any of the respective embodiments described.

In some of any of the embodiments described herein, materials which sink to the bottom are removed (e.g., by removing sediment), and substantially all other materials are collected as a first fraction according to any of the respective embodiments described herein.

In some of any of the embodiments described herein, the separation process comprises removing substantially all of the material from the liquid (e.g., both the fraction of low-density materials and the fraction of high-density materials), such that the liquid can be reused to separate more waste material according to specific gravity. Removal from the liquid can be for example, by skimming floating material from a surface, removing sedimented material, and/or filtering out material which sinks below a surface of the liquid but does not sink to the bottom.

The specific gravity of the liquid may be selected in accordance with the materials which are desired to be included within a fraction of low-density materials (e.g., first fraction) and/or with the materials which are desired to be included within a fraction of high-density materials (e.g., second fraction), for example, in accordance with whether it is desired for lignocellulose to be included primarily in the first fraction or in the second fraction.

Without being bound by any particular theory, it is believed that that separation by contacting waste material with a liquid may be readily performed using wet waste material (e.g., waste material that has not been dried), whereas wet waste material may pose an obstacle to other separation techniques, for example, by resulting in fragments of different types of material sticking to one another.

In some embodiments of any of the embodiments relating to utilization of a liquid, at least two distinct liquids are utilized, and the waste material is thereby separated into at least three fractions.

In some embodiments, the separation process comprises contacting the waste material with a first aqueous liquid, to thereby obtain a first and second fraction as described herein, and further comprises contacting at least one (optionally only one) of the first fraction and the second fraction with a second aqueous liquid, thereby obtaining a third fraction of low-density materials which do not sink in either of the first or second aqueous liquids, a fourth fraction of intermediate-density materials which sink in one of the aqueous liquids (e.g., whichever liquid has a lower specific gravity), and a fifth fraction of high-density materials which sink in both the first and second aqueous liquids.

The second aqueous liquid is optionally selected such that a portion of the fraction contacted therewith sinks. In some embodiment of any of the embodiments relating to a first and second aqueous liquid, the second aqueous liquid has a different specific gravity than the first aqueous liquid.

In some embodiment of any of the embodiments relating to a first and second aqueous liquid, specific gravities of the first and second aqueous liquids differ by at least 0.01. In some embodiments, specific gravities of the first and second aqueous liquids differ by at least 0.02. In some embodiments, specific gravities of the first and second aqueous liquids differ by at least 0.03. In some embodiments, specific gravities of the first and second aqueous liquids differ by at least 0.05. In some embodiments, specific gravities of the first and second aqueous liquids differ by at least 0.07. In some embodiments, specific gravities of the first and second aqueous liquids differ by at least 0.1. In some embodiments, specific gravities of the first and second aqueous liquids differ by at least 0.15. In some embodiments, specific gravities of the first and second aqueous liquid differ by at least 0.2.

Herein, the phrases “materials which do not sink in either of the first or second aqueous liquids”, “materials which do not sink in either of said aqueous liquids” and the like, encompass materials which do not sink in whichever of the aqueous liquids has the lowest specific gravity, without requiring any determination of the behavior of the materials in a liquid with a higher specific gravity.

Similarly, herein, the phrase “materials which sink in both the first and second aqueous liquids”, “materials which sink in both of said aqueous liquids” and the like, encompass materials which sink in whichever of the aqueous liquids has the highest specific gravity, without requiring any determination of the behavior of the materials in a liquid with a lower specific gravity.

It is to be understood that the phrases “third fraction”, “fourth fraction” and “fifth fraction” merely indicate that the separation process comprises at least two separations which result in at least three fractions, and does not necessarily mean that the fraction is different than a “first fraction” or “second fraction” described herein.

It is also to be understood that the phrases “first fraction” and “second fraction” indicate that those two fractions are obtained during the separation process, and does not necessarily mean that the separation process does not comprise further separation into three or more fractions, as described herein. Such further separation may be before and/or after the separation into first and second fractions.

In some embodiment of any of the embodiments described herein, a first fraction of low-density materials obtained using a first aqueous liquid is contacted with a second aqueous liquid, wherein the second aqueous liquid has a lower specific gravity than the first aqueous liquid. In some such embodiments, the second fraction of high-density materials is identical to the fifth fraction of high-density materials, and the first fraction of low-density materials is separated into the third fraction of low-density materials and the fourth fraction of intermediate-density materials. It is to be appreciated that in such embodiments, the fourth fraction may be considered the fraction of high-density materials with respect to the second aqueous liquid.

In some embodiment of any of the embodiments described herein, a second fraction of high-density materials obtained using a first aqueous liquid is contacted with a second aqueous liquid, wherein the second aqueous liquid has a higher specific gravity than the first aqueous liquid. In some such embodiments, the first fraction of low-density materials is identical to the third fraction of low-density materials, and the second fraction of high-density materials is separated into the fifth fraction of high-density materials and the fourth fraction of intermediate-density materials. It is to be appreciated that in such embodiments, the fourth fraction of intermediate-density materials may be considered the fraction of low-density materials with respect to the second aqueous liquid.

In some embodiment of any of the embodiments described herein relating to two aqueous liquids having different specific gravities, use of the liquid with a higher specific gravity as the first aqueous liquid and use of the liquid with a higher specific gravity as the second aqueous liquid result in substantially the same fractions, that is the fractions are not substantially affected by the order in which the liquids are utilized.

In some embodiment of any of the embodiments described herein relating to two aqueous liquids having different specific gravities, a specific gravity of one of the aqueous liquids is no more than 1.01, the liquid optionally being water. In some such embodiments, a specific gravity of the other of the two aqueous liquids is at least 1.03 (e.g., according to any of the embodiments described herein relating to a liquid with such a specific gravity). In some such embodiments, a specific gravity of the other of the two aqueous liquids is at least 1.05 (e.g., according to any of the embodiments described herein relating to a liquid with such a specific gravity). In some such embodiments, a specific gravity of the other of the two aqueous liquids is at least 1.07 (e.g., according to any of the embodiments described herein relating to a liquid with such a specific gravity). In some such embodiments, a specific gravity of the other of the two aqueous liquids is at least 1.10 (e.g., according to any of the embodiments described herein relating to a liquid with such a specific gravity). In some such embodiments, a specific gravity of the other of the two aqueous liquids is at least 1.15 (e.g., according to any of the embodiments described herein relating to a liquid with such a specific gravity). In some such embodiments, a specific gravity of the other of the two aqueous liquids is at least 1.20 (e.g., according to any of the embodiments described herein relating to a liquid with such a specific gravity).

In some embodiment of any of the embodiments described herein relating to two aqueous liquids having different specific gravities, a specific gravity of one of the aqueous liquids is no more than 1.00, the liquid optionally being water. In some such embodiments, a specific gravity of the other of the two aqueous liquids is at least 1.03 (e.g., according to any of the embodiments described herein relating to a liquid with such a specific gravity). In some such embodiments, a specific gravity of the other of the two aqueous liquids is at least 1.05 (e.g., according to any of the embodiments described herein relating to a liquid with such a specific gravity). In some such embodiments, a specific gravity of the other of the two aqueous liquids is at least 1.07 (e.g., according to any of the embodiments described herein relating to a liquid with such a specific gravity). In some such embodiments, a specific gravity of the other of the two aqueous liquids is at least 1.10 (e.g., according to any of the embodiments described herein relating to a liquid with such a specific gravity). In some such embodiments, a specific gravity of the other of the two aqueous liquids is at least 1.15 (e.g., according to any of the embodiments described herein relating to a liquid with such a specific gravity). In some such embodiments, a specific gravity of the other of the two aqueous liquids is at least 1.20 (e.g., according to any of the embodiments described herein relating to a liquid with such a specific gravity).

In some embodiment of any of the embodiments described herein relating to two aqueous liquids having different specific gravities, the aqueous liquid having a higher specific gravity is an aqueous salt solution according to any of the respective embodiments described herein.

In some of any of the embodiments described herein, a fourth fraction of intermediate-density materials (according to any of the respective embodiments described herein) is the separated lignocellulose.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.05 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.06 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.07 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.08 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.09 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.10 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.11 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.12 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.13 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.14 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.15 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.175 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of at least 1.20 (in which the intermediate-density materials do not sink), as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.03 (in which the intermediate-density materials sink) and in a liquid having a specific gravity of no more than 1.30 (in which the intermediate-density materials do not sink), for example, in a range of from 1.03 to 1.30, as described in any of the embodiments herein pertaining to such liquids. In some such embodiments, the specific gravity of the liquid in which the intermediate-density materials sink is no more than 1.02 (according to any of the embodiments herein pertaining to such a liquid). In some such embodiments, the specific gravity of the liquid in which the intermediate-density materials sink is no more than 1.01 (according to any of the embodiments herein pertaining to such a liquid). In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In some of any of the embodiments described herein, a fourth fraction of intermediate-density materials which is the separated lignocellulose is obtained by separating material (waste material or a fraction thereof) in a liquid having a specific gravity of no more than 1.01 (in which the intermediate-density materials sink) and in a liquid having a specific gravity of at least 1.03 (in which the intermediate-density materials do not sink), as described in any of the embodiments herein pertaining to such liquids. In some such embodiments, the specific gravity of the liquid in which the intermediate-density materials sink is no more than 1.00 (according to any of the embodiments herein pertaining to such a liquid). In some such embodiments, the liquid in which the intermediate-density materials sink is water.

In general, a larger difference between specific gravities of two liquids used for separation will result in a greater amount of material in a fraction of intermediate-density materials, which may be generally associated with greater yields of lignocellulose but in lower selectivity (e.g., more materials other than lignocellulose as impurities; whereas a smaller difference between specific gravities of two liquids used for separation will result in a smaller amount of material in a fraction of intermediate-density materials, which may be associated with smaller yields of lignocellulose but in greater selectivity (e.g., lower concentrations of impurities). The skilled person will be capable of selecting appropriate specific gravities based on such considerations.

In some embodiments of any of the embodiments described herein, the method provides at least one fraction enriched in material having a specific gravity within a pre-selected range, and the liquid is selected in accordance with the pre-selected range (e.g., selection of a suitable concentration for an aqueous salt solution, as discussed in further detail herein).

In some embodiments of any of the embodiments described herein, the fraction(s) contains at least 90 weight percents of material having a specific gravity within a pre-selected range. In some embodiments, the fraction(s) contains at least 95 weight percents of material having a specific gravity within a pre-selected range. In some embodiments, the fraction(s) contains at least 98 weight percents of material having a specific gravity within a pre-selected range. In some embodiments, the fraction(s) contains at least 99 weight percents of material having a specific gravity within a pre-selected range. Any value between 90 and 99.9 weight percents is also contemplated according to these embodiments.

A pre-selected range for the specific gravity may optionally be characterized by an upper limit and a lower limit, or alternatively, the range may optionally be an open-ended range, for example, characterized by an upper limit with no lower limit, or by a lower limit with no upper limit.

In some embodiments of any of the embodiments described herein, the pre-selected range for a first fraction of low-density materials according to any of the respective embodiments described herein is no more than 1.25, that is, the upper limit of the pre-selected range is no more than 1.25, such that the entire range is no more than 1.25. In some embodiments, the pre-selected range is no more than 1.225. In some embodiments, the pre-selected range is no more than 1.20. In some embodiments, the pre-selected range is no more than 1.175. In some embodiments, the pre-selected range is no more than 1.15. In some embodiments, the pre-selected range is no more than 1.125. In some embodiments, the pre-selected range is no more than 1.10. In some embodiments, the pre-selected range is no more than 1.075. In some embodiments, the pre-selected range is no more than 1.05. In some embodiments, the pre-selected range is no more than 1.025. In some embodiments, the pre-selected range is no more than 1.00.

In some embodiments of any of the embodiments described herein, the pre-selected range for a third fraction of low-density materials according to any of the respective embodiments described herein is no more than 1.25, that is, the upper limit of the pre-selected range is no more than 1.25, such that the entire range is no more than 1.25. In some embodiments, the pre-selected range is no more than 1.225. In some embodiments, the pre-selected range is no more than 1.20. In some embodiments, the pre-selected range is no more than 1.175. In some embodiments, the pre-selected range is no more than 1.15. In some embodiments, the pre-selected range is no more than 1.125. In some embodiments, the pre-selected range is no more than 1.10. In some embodiments, the pre-selected range is no more than 1.075. In some embodiments, the pre-selected range is no more than 1.05. In some embodiments, the pre-selected range is no more than 1.025. In some embodiments, the pre-selected range is no more than 1.00.

In some of any of the embodiments described herein, the waste material is stirred in the liquid, for example, by rotation of at least one paddle (e.g., rotation of a paddle wheel). Stirring is optionally selected to be sufficiently vigorous to facilitate separation of different types of material (which may be stuck to one another, for example), while being sufficiently gentle to allow separation of materials in the liquid.

In some of any of the embodiments described herein, the stirring comprises perturbation (e.g., rotation, vibration, agitation) at a frequency of 120 per minute or less. In some embodiments, stirring comprises perturbation at a frequency of 60 per minute or less. In some embodiments, stirring comprises perturbation at a frequency of 30 per minute or less. In some embodiments, stirring comprises perturbation at a frequency of 20 per minute or less. In some embodiments, stirring comprises perturbation at a frequency of 10 per minute or less.

Although embodiments comprising one or two cycles of separating materials according to specific gravity are described herein explicitly, it is to be understood that in some of any of the embodiments described herein, the method comprises more than two cycles of separating materials according to specific gravity.

In addition, it is to be understood that each cycle may be effected with a liquid (e.g., an aqueous salt solution) which is the same or different than a liquid (e.g., an aqueous salt solution) used in another cycle, and that each cycle may independently comprise separating a fraction of high-density materials (e.g., materials which sink in the liquid) and/or removing a fraction of low-density materials (e.g., materials which float in the liquid).

Liquids Utilized in Separation Process:

As described herein, the liquid utilized in a separation process according to any of the respective embodiments described herein may be a pure liquid, a solution, or a suspension. In some embodiments of any of the embodiments described herein, the liquid is an aqueous liquid.

As used herein, the phrase “aqueous liquid” refers to a liquid in which at least 50 weight percents of the liquid compound(s) therein (e.g., excluding solid materials suspended and/or dissolved in the liquid) is water. In some embodiments, at least 60 weight percents is water. In some embodiments, at least 70 weight percents is water. In some embodiments, at least 80 weight percents is water. In some embodiments, at least 90 weight percents is water. In some embodiments, at least 95 weight percents is water. In some embodiments, at least 98 weight percents is water. In some embodiments, at least 99 weight percents is water. In some embodiments, the liquid component substantially consists of water.

In some embodiments of any of the embodiments described herein, the liquid is a solution, for example, an aqueous solution. Suitable solutes for a solution (e.g., an aqueous solution) include water-soluble salts, that is, any compound which form ions in water (e.g., sodium chloride, potassium chloride, sodium bromide, potassium bromide, calcium chloride, calcium nitrate, potassium carbonate) and water-soluble carbohydrates (e.g., glucose, sucrose, lactose, fructose).

In some embodiments of any of the embodiments described herein, the solute is a salt, that is, the liquid is an aqueous salt solution (solution of ions). In some embodiments the salt comprises sodium chloride. The sodium chloride may optionally be substantially pure. Alternatively, the sodium chloride is mixed with other salts, for example, as in sea salt.

In some embodiments of any of the embodiments described herein, the liquid comprises sea water (e.g., sea water diluted with fresh water and/or concentrated sea water, that is, sea water from which a portion of the water has been removed). In some embodiments, the liquid consists essentially of sea water.

In some embodiments of any of the embodiments described herein, the liquid is a suspension, for example, an aqueous suspension. Suitable suspended materials for a suspension include water-insoluble salts and/or metallic substances, such as, for example, calcium carbonate, iron powder and ferrosilicon (FeSi). In some embodiments, the suspended material is magnetic, which facilitates removal its removal from separated waste materials (e.g., for reuse).

The specific gravity of a solution or a suspension can be finely controlled in accordance with the separation requirements, by controlling the concentration of the solute or suspended material.

Thus, for example, if a relatively high specific gravity is desired for a fraction of high-density materials, a solution or suspension with a relatively high specific gravity (yet lower than that of the materials to be included in the fraction of high-density materials) is to be used, and therefore, a high concentration of the solute or suspended material is included.

If a relatively low specific gravity (e.g., below that of water) is desired for a fraction of low-density materials, a solution or suspension with a relatively low specific gravity (yet higher than that of the materials to be included in the fraction of low-density materials) is to be used, and therefore, a low concentration (optionally zero) of the solute or suspended material is included.

In some embodiments of any of the embodiments described herein, a specific gravity of a liquid is in a range of from 1.00 to 2.50, preferably in a range of from 1.00 to 1.50.

In some embodiments of any of the embodiments described herein, the specific gravity of a liquid (e.g., a liquid in which the intermediate-density materials according to any of the respective embodiments described herein do not sink) is at least 1.20, for example, in a range of from 1.20 to 1.50. A specific gravity of at least 1.20 may be suitable, for including many or even most organic materials in a fraction of low-density materials, while including some organic materials (e.g., high-density polymeric materials) in a fraction of high-density materials. In some embodiments, the specific gravity of a liquid is at least 1.25. In some embodiments, the specific gravity of a liquid is at least 1.30. In some embodiments, the specific gravity of a liquid is at least 1.35. In some embodiments, the specific gravity of a liquid is at least 1.40. In some embodiments, the specific gravity of a liquid is at least 1.45.

In some embodiments of any of the embodiments described herein, the specific gravity of a liquid (e.g., a liquid in which the intermediate-density materials according to any of the respective embodiments described herein do not sink) is at least 1.01, for example, in a range of from 1.01 to 1.20. A specific gravity in a range of 1.01 to 1.20 may be suitable, for including many or even most animal materials and plant materials (e.g., lignocellulose) in a fraction of low-density materials, while including many synthetic polymers (e.g., high-density polymeric materials), such as polyethylene terephthalate (PET), polytetrafluoroethylene (PTFE)) and polyvinyl chloride (PVC), in a fraction of high-density materials.

In some embodiments of any of the embodiments described herein, the specific gravity of the liquid is no more than about 1.25 (e.g., about the specific gravity of a saturated aqueous solution of sea salt). In some embodiments, the specific gravity is no more than 1.20. In some embodiments, the specific gravity is no more than 1.15.

In some embodiments of any of the embodiments described herein, the specific gravity of a liquid (e.g., a liquid in which the intermediate-density materials according to any of the respective embodiments described herein do not sink) is at least 1.05. In some embodiments, the specific gravity is in a range of from 1.05 to 1.25. In some embodiments, the specific gravity is in a range of from 1.05 to 1.20. In some embodiments, the specific gravity is in a range of from 1.05 to 1.15.

In some embodiments of any of the embodiments described herein, the specific gravity of a liquid (e.g., a liquid in which the intermediate-density materials according to any of the respective embodiments described herein do not sink) is at least 1.06. In some embodiments, the specific gravity is in a range of from 1.06 to 1.25. In some embodiments, the specific gravity is in a range of from 1.06 to 1.20. In some embodiments, the specific gravity is in a range of from 1.06 to 1.15.

In some embodiments of any of the embodiments described herein, the specific gravity of a liquid (e.g., a liquid in which the intermediate-density materials according to any of the respective embodiments described herein do not sink) is at least 1.07 (e.g., an aqueous sodium chloride solution at a concentration of about 10 weight percents). In some embodiments, the specific gravity is in a range of from 1.07 to 1.25. In some embodiments, the specific gravity is in a range of from 1.07 to 1.20. In some embodiments, the specific gravity is in a range of from 1.07 to 1.15.

In some embodiments of any of the embodiments described herein, the specific gravity of a liquid (e.g., a liquid in which the intermediate-density materials according to any of the respective embodiments described herein do not sink) is at least 1.08. In some embodiments, the specific gravity is in a range of from 1.08 to 1.25. In some embodiments, the specific gravity is in a range of from 1.08 to 1.20. In some embodiments, the specific gravity is in a range of from 1.08 to 1.15.

In some embodiments of any of the embodiments described herein, the specific gravity of a liquid (e.g., a liquid in which the intermediate-density materials according to any of the respective embodiments described herein do not sink) is at least 1.09. In some embodiments, the specific gravity is in a range of from 1.09 to 1.25. In some embodiments, the specific gravity is in a range of from 1.09 to 1.20. In some embodiments, the specific gravity is in a range of from 1.09 to 1.15.

In some embodiments of any of the embodiments described herein, the specific gravity of a liquid (e.g., a liquid in which the intermediate-density materials according to any of the respective embodiments described herein do not sink) is at least 1.10. In some embodiments, the specific gravity is in a range of from 1.10 to 1.25. In some embodiments, the specific gravity is in a range of from 1.10 to 1.20. In some embodiments, the specific gravity is in a range of from 1.10 to 1.15.

In some embodiments of any of the embodiments described herein, the specific gravity of a liquid (e.g., a liquid in which the intermediate-density materials according to any of the respective embodiments described herein do not sink) is at least 1.11 (e.g., an aqueous sodium chloride solution at a concentration of about 15 weight percents). In some embodiments, the specific gravity is in a range of from 1.11 to 1.25. In some embodiments, the specific gravity is in a range of from 1.11 to 1.20.

In some embodiments of any of the embodiments described herein, the specific gravity of a liquid (e.g., a liquid in which the intermediate-density materials according to any of the respective embodiments described herein do not sink) is at least 1.12. In some embodiments, the specific gravity is in a range of from 1.12 to 1.25. In some embodiments, the specific gravity is in a range of from 1.12 to 1.20.

In some embodiments of any of the embodiments described herein, the specific gravity of a liquid (e.g., a liquid in which the intermediate-density materials according to any of the respective embodiments described herein do not sink) is at least 1.13. In some embodiments, the specific gravity is in a range of from 1.13 to 1.25. In some embodiments, the specific gravity is in a range of from 1.13 to 1.20.

In some embodiments of any of the embodiments described herein, the specific gravity of a liquid (e.g., a liquid in which the intermediate-density materials according to any of the respective embodiments described herein do not sink) is at least 1.14. In some embodiments, the specific gravity is in a range of from 1.14 to 1.25. In some embodiments, the specific gravity is in a range of from 1.14 to 1.20.

In some embodiments of any of the embodiments described herein, the specific gravity of a liquid (e.g., a liquid in which the intermediate-density materials according to any of the respective embodiments described herein do not sink) is at least 1.15 (e.g., an aqueous sodium chloride solution at a concentration of about 20 weight percents). In some embodiments, the specific gravity is in a range of from 1.15 to 1.25. In some embodiments, the specific gravity is in a range of from 1.15 to 1.20.

In some embodiments of any of the embodiments described herein, the specific gravity of a liquid (e.g., a liquid in which the intermediate-density materials according to any of the respective embodiments described herein do not sink) is at least 1.175. In some embodiments, the specific gravity is in a range of from 1.175 to 1.25. In some embodiments, the specific gravity is in a range of from 1.175 to 1.20.

In some embodiments of any of the embodiments described herein, the specific gravity of a liquid (e.g., a liquid in which the intermediate-density materials according to any of the respective embodiments described herein do not sink) is at least 1.20. In some embodiments, the specific gravity is in a range of from 1.20 to 1.25.

In some embodiments of any of the embodiments described herein, the specific gravity of a liquid (e.g., a liquid in which the intermediate-density materials according to any of the respective embodiments described herein sink) is approximately 1.03 or less, for example, in a range of from 1.01 to 1.03. A specific gravity in a range may conveniently and inexpensively be obtained, for example, using sea water or diluted sea water, as sea water has a specific gravity in a range of from 1.02 to 1.03, typically approximately 1.025.

In general, liquids with relatively low specific gravities (e.g., up to 1.25, up to 1.20) are relatively convenient to prepare and use, they may readily be obtained from solutions of common and inexpensive materials. For example, specific gravities of aqueous sodium chloride solutions range from 1.00 to about 1.20, depending on concentration.

In some embodiments of any of the embodiments described herein, specific gravities of at least 1.20, optionally at least 1.25, are obtained using high density water-soluble salts such as calcium salts, magnesium salts, transition metal salts, bromide salts and/or using suspensions.

Without being bound by any particular theory, it is believed that contact of waste material with a salt solution inhibits microbial (e.g., bacterial) survival and/or activity in the obtained fractions and/or separated materials (in addition to facilitating the separation process). Such inhibition is comparable to preservation of food in salt water (e.g., pickling). Such inhibition may for example, enhance hygiene and/or reduce malodor of fractions and/or separated materials, thereby and facilitating their handling and/or storage.

In some embodiments of any of the embodiments described herein, a concentration of salt in a solution is selected to be capable of inhibiting microbial (e.g., bacterial) survival and/or activity in waste material contacted with the solution, and/or in fractions, separated material and/or processed material (e.g., as described herein) derived therefrom.

In some embodiments, separated lignocellulose is contacted with a non-saline liquid (e.g., water), optionally a liquid used for a second separation stage, to reduce salt concentrations prior to exposure to microorganisms, in order to minimize deleterious effects of salt on the microorganisms.

Furthermore, as exemplified herein, salt solutions (e.g., at concentrations of above about 10 weight percents) result in release of carbohydrates from biomass, indicating considerable disruption of the structure thereof (e.g., rupture of cells).

Without being bound by any particular theory, it is believed that such disruption can advantageously facilitate processing of separated lignocellulose by breaking down lignocellulose (e.g., by hydrolysis and/or disruption of noncovalent bonds within lignocellulose) and/or cells, and rendering carbohydrates more available for metabolism by microorganisms.

On the other hand, it is believed that such release of carbohydrates (e.g., into a liquid used for separation) may represent a loss of carbohydrates which could have been utilized for metabolism by microorganisms. Such a loss may optionally be minimized by avoiding exposure to a salt solution for an excessive time period, such that carbohydrates may become exposed by the effects of the salt solution, without diffusing out of the lignocellulosic biomass.

In some embodiments of any of the embodiments described herein, the concentration of salt (e.g., sodium chloride, sea salt) in a salt solution (e.g., aqueous salt solution) is at least 3 weight percents. In some embodiments, the concentration of salt is in a range of from 3 to 35 weight percents. In some embodiments, the concentration of salt is in a range of from 3 to 30 weight percents. In some embodiments, the concentration of salt is in a range of from 3 to 25 weight percents.

In some embodiments of any of the embodiments described herein, the concentration of salt (e.g., sodium chloride, sea salt) in a salt solution (e.g., aqueous salt solution) is at least 5 weight percents. In some embodiments, the concentration of salt is in a range of from 5 to 35 weight percents. In some embodiments, the concentration of salt is in a range of from 5 to 30 weight percents. In some embodiments, the concentration of salt is in a range of from 5 to 25 weight percents.

In some embodiments of any of the embodiments described herein, the concentration of salt (e.g., sodium chloride, sea salt) in a salt solution (e.g., aqueous salt solution) is at least 10 weight percents. In some embodiments, the concentration of salt is in a range of from 10 to 35 weight percents. In some embodiments, the concentration of salt is in a range of from 10 to 30 weight percents. In some embodiments, the concentration of salt is in a range of from 10 to 25 weight percents.

In some embodiments of any of the embodiments described herein, the concentration of salt (e.g., sodium chloride, sea salt) in a salt solution (e.g., aqueous salt solution) is at least 15 weight percents. In some embodiments, the concentration of salt is in a range of from 15 to 35 weight percents. In some embodiments, the concentration of salt is in a range of from 15 to 30 weight percents. In some embodiments, the concentration of salt is in a range of from 15 to 25 weight percents.

In some embodiments of any of the embodiments described herein, the concentration of salt (e.g., sodium chloride, sea salt) in a salt solution (e.g., aqueous salt solution) is at least 20 weight percents. In some embodiments, the concentration of salt is in a range of from 20 to 35 weight percents. In some embodiments, the concentration of salt is in a range of from 20 to 30 weight percents. In some embodiments, the concentration of salt is in a range of from 20 to 25 weight percents.

Without being bound by any particular theory, it is believed that contact of waste material and/or a fraction derived therefrom with a salt solution comprising salt concentrations of at least 10 weight percents, especially at least 15 weight percents, and most especially at least 20 weight percents, is particularly effective at inhibiting microbial (e.g., bacterial) survival and/or activity not only in material contacted with the solution, but also at inhibiting microbial (e.g., bacterial) survival and/or activity in separated material and/or processed material (e.g., as described herein) derived therefrom, that is, residual salt remaining in the separated material and/or processed material (after the material has been removed from the salt solution) can effectively inhibit microbial survival and/or activity long after the separation according to specific gravity has been completed.

It is to be appreciated that cellulose and other compounds from animal material or plant material (e.g., lignin) are characterized by a specific gravity of approximately 1.5, but that animal materials and plant materials typically exhibit considerably lower specific gravities as a result of porosity (for, example, the voids in wood, which reduce the specific gravity of most wood to less than 1) and/or a considerable amount of water therein (which results in a specific gravity close to 1). Thus, a specific gravity of many materials is indicative of its water content and/or porosity.

Sonication:

In some embodiments of any of the embodiments described herein, irradiation by sound or ultrasound is further effected. In some such embodiments, irradiating of waste material is performed with the waste material in a liquid, thereby allowing the sound or ultrasound to propagate in the liquid and interact with the waste material. In some embodiments, irradiation by sound or ultrasound is effected by irradiating waste material in a liquid selected such that a portion of the waste material sinks in the liquid (according to any of the embodiments described herein pertaining to such a liquid), for example, an aqueous solution described herein.

A sound wave is a pressure wave, typically a longitudinal pressure wave.

Ultrasound is a sound wave (pressure wave) at a frequency which is generally above the upper range of human hearing (e.g., above 18 kHz). Below about 1 MHz, ultrasound waves are called “low frequency ultrasound”. At this range, energy is transferred at a high power level and is able to modify a liquid in which it propagates, for example, by disrupting the liquid bulk to create cavitation and/or acoustic streaming, two phenomena with significant macroscopic effects.

In preferred embodiments, the irradiation is ultrasound, that is, at an ultrasound frequency.

Irradiation according to alternative embodiments in which the sound wave is not an ultrasound wave are described herein simply by the terms “sound” and “sound wave”. Similarly, ultrasound and non-ultrasound sound waves are collectively described herein using phrases such as “sound or ultrasound”. However, this should not be construed as a suggestion that ultrasound waves are not a type of sound wave.

Acoustic streaming ensues from the dissipation of acoustic energy which permits the gradients in momentum, and thereby the fluid currents. The speed gained by a liquid allows a better convection heat transfer coefficient at solid-liquid interface, and can induce turbulence and promote heat transfer.

In cavitation, micron-size bubbles are formed. The stability of the cavitation bubbles depends on the parameters of the sound or ultrasound wave (e.g., the intensity, pulse energy and/or duty cycle). While the following discussion of cavitation is described with a particular emphasis to sound or ultrasound intensity, the ordinarily skilled person would appreciate that similar discussion can be formulated also with respect to other sound or ultrasound parameters including, without limitation, the pulse energy and/or duty cycle. Further, cavitation can be formed according to some embodiments of the present invention by other means, including, without limitation, by hydrodynamic means.

Already at relatively low sound intensities (for example, from about 1 W/cm² to about 3 W/cm²) the bubbles do not perish but exhibit stable volume and/or shape oscillations. This type of cavitation is denoted as “stable” or “non-inertial” cavitation. In stable cavitation, the bubbles typically oscillate about some equilibrium size for many acoustic cycles.

When the sound or ultrasound intensity is increased and exceeds a certain limit, known as the cavitation threshold, the nature of cavitation changes dramatically which results in the bubbles becoming unstable. Within a fraction of a sound cycle they show rapid growth followed by a violent implosive collapse. More specifically, as the bubble contracts from its maximum to minimum radius, the surrounding liquid gains an inwardly directed momentum. At sufficiently high sound or ultrasound intensities, the gained momentum is sufficiently high such that the rising pressure within the bubble is unable to resist the liquid coming in, and the bubble collapses. Cavitation which shows this violent bubble behavior is referred to as “transient cavitation” or “inertial cavitation.” The cavitation is “inertial” in the sense that the cause of the collapse is the inertia from the liquid. The cavitation threshold is typically, but not necessarily, about 10 W/cm².

In inertial cavitation, during the collapse, the speed of a gas-liquid interface may become very high and at sufficiently high sound or ultrasound intensities becomes supersonic, in which case an outwardly propagating shock wave is generated in the liquid. During the time at which the bubble approaches its minimum radius, the pressure in the bubble increases significantly, typically to above 100 or above 1000 MPa. Consequently, the temperature is also increased and may reach more than 2000 K. At high temperatures, free radicals occur. For example, in water, inertial cavitation may lead to the formation of H and OH free radicals.

In inertial cavitation, the bubble may affect solid surfaces contacting the bubble already during the expansion phase of the bubble's oscillation. For example, when a bubble is trapped in a capillary, at sufficiently high sound or ultrasound intensities the bubble can rupture the walls of the capillary during the expansion phase.

In some embodiments of this aspect of the present invention, separating materials according to specific gravity is facilitated by irradiation of waste material by sound or ultrasound prior to and/or concurrent with the separation.

Without being bound by any particular theory, it is believed that sound or ultrasound waves, especially under cavitation conditions, enhance separation of different materials (e.g., materials having different specific gravities) which are bound to one another in the waste material (e.g., by covalent and/or non-covalent bonds such as hydrogen bonds), and break down large molecules (e.g., polymers) into smaller molecules (e.g., smaller polymers). It is further believed that that sound or ultrasound waves, especially under cavitation conditions, degrade individual materials by reducing a degree of internal bonding, for example, by hydrogen bonds, thereby facilitating suspension and/or dissolution of materials.

In some embodiments, sound or ultrasound enhances separation of composite materials into components thereof, including separation of natural composite materials (e.g., lignocellulose) and/or synthetic composite materials (e.g., composites with two or more polymeric materials and/or polymer-metal composites).

For example, it was found by the present inventors that the sound or ultrasound wave can promote separation of hemicellulose and lignin from lignocellulose, and degrade cellulose and polyolefins.

In some embodiments, sound or ultrasound enhances suspension and/or dissolution of cellulose in an aqueous liquid described herein.

In various exemplary embodiments of the invention at least one parameter of the sound or ultrasound is selected such as to generate an inertial cavitation condition in the waste material.

In some embodiments, at least one parameter of the sound or ultrasound is selected such as to generate a shock wave in a liquid containing the waste material (optionally a liquid selected such that a portion of the waste material sinks in the liquid, according to any of the respective embodiments described herein).

In some embodiments, at least one parameter of the sound or ultrasound is selected such as to induce rapture of capillaries in the waste material during the expansion phase of the cavitation bubbles.

In various exemplary embodiments of the invention at least one parameter of the sound or ultrasound is selected such as to generate an inertial cavitation condition in the waste material, in some embodiments, at least one parameter of the sound or ultrasound is selected such that the collapse of the bubbles of the inertial cavitation results in microjets moving at a speed of at least 500 m/sec, in some embodiments, at least one parameter of the sound or ultrasound is selected such as to generate a shock wave in a liquid containing the waste material, and in some embodiments, at least one parameter of the sound or ultrasound is selected such as to induce rupture of capillaries in the waste material during the expansion phase of the cavitation bubbles.

In a preferred embodiment, at least one parameter of the sound or ultrasound is selected such as to generate an inertial cavitation condition that induces formation of free radicals, for example, carbon-centered free radicals in the waste material, and/or hydrogen and/or hydroxyl free radicals in a liquid containing the waste material.

In some embodiments of any of the embodiments described herein pertaining to sound or ultrasound irradiation, the sound or ultrasound is at a frequency of at least 20 kHz (i.e., ultrasound), optionally in a range of from 20 kHz to 2 MHz, optionally in a range of from 20 kHz, optionally in a range of from 20 kHz to 200 kHz, and optionally in a range of from 20 kHz to 100 kHz.

In some embodiments of any of the embodiments described herein pertaining to ultrasound irradiation, the ultrasound is at a frequency of at least 40 kHz, optionally in a range of from 40 kHz to 2 MHz, optionally in a range of from 40 kHz, optionally in a range of from 40 kHz to 200 kHz, and optionally in a range of from 40 kHz to 100 kHz.

In some embodiments of any of the embodiments described herein pertaining to sound or ultrasound irradiation, the sonicated material receives an average intensity of at least 1 W/cm². In some embodiments, the average intensity is at least 3 W/cm². In some embodiments, the average intensity is at least 10 W/cm².

In some embodiments, irradiation of material by sound or ultrasound is effected for a time period in a range of from 1 to 60 minutes.

Microbial Digestion:

In some embodiments of any of the embodiments described herein, processing a separated lignocelluloses to produce biogas or ethanol is effected by subjecting at least one fraction which comprises separated lignocellulose or which is lignocelluloses-enriched, as described herein, to digestion by microorganisms and/or by enzymes related thereto, collectively referred to herein as “microbial digestion”.

Herein, the term “microbial digestion” refers to use of organisms, preferably microorganisms, to metabolize at least a portion of a material subjected to digestion into different material(s), for example, compounds not present in the original material in an isolated form. A microbial digestion can involve processes performed by the organism as a whole or by enzymes related to the organism, which can be either isolated from the microorganism or not.

In some of any of the embodiments described herein, the microbial digestion is an anaerobic digestion, performed under conditions in which oxygen is absent.

The term “microorganisms” as used herein includes bacteria, archaea, fungi, protozoa, and other microorganisms known to one of skill in the art to digest lignocellulosic biomass to produce biogas.

An anaerobic digestion is also referred to herein and in the art as “fermentation”, when effected by yeast and/or related enzymes, so as to produce ethanol from soluble carbohydrates (sugars).

As discussed hereinabove, microbial digestion of lignocellulosic biomass is beneficially performed for producing a biogas or ethanol (e.g., bioethanol), as described and defined hereinabove.

A skilled person will be capable of selecting a microbial digestion process according to a desired product, for example, by selecting one or more appropriate organisms, by controlling conditions under which the microbial digestion proceeds, and/or by selecting a suitable technique for extracting a desired product, utilizing techniques known in the art.

Production of Biogas:

In some of any of the embodiments described herein, a method as described herein is used for producing biogas such as carbon dioxide and/or methanol, and is effected by anaerobic microbial digestion using any of the microorganisms known in the art to effect biogas production from biomass. Typically, biogas production by microbial digestion is effected by a combination of microorganisms, which can be introduced into a single bioreactor, or by means of a plurality of reactors, each comprising different one or more of the microorganisms participating in the production of biogas.

Typically, an anaerobic digestion process generally begins with bacterial hydrolysis of the input materials, namely, lignocellulose. Insoluble carbohydrates such as cellulose and hemicellulose, are broken down to soluble derivatives that become available for other bacteria. During the hydrolysis stage, simple sugars, amino acids, and fatty acids are produced.

Acidogenic bacteria then convert the sugars and amino acids into carbon dioxide, hydrogen sulfide, ammonia, and organic acids, typically volatile fatty acids. The third stage of anaerobic digestion is acetogenesis, in which the molecules produced through the acidogenesis phase are further digested by acetogens to produce mainly acetic acid, as well as carbon dioxide and hydrogen.

Finally, methanogens convert the intermediate products of any of the preceding stages into methane, carbon dioxide, and water.

An exemplary method for producing biogas by microbial digestion of a separated lignocellulose as described herein is as follows:

Microbially digesting the separated lignocellulose comprises digesting the lignocellulose with one or more microorganisms for a time sufficient to anaerobically digest the lignocellulose so as to produce biogas, such as methane and carbon dioxide. The separated lignocellulose as described herein is added to a bioreactor which optionally contains one or more exogenous microorganisms capable of digesting the lignocelluloses, or to which the one or more exogenous microorganisms are added. Alternatively, exogenous microorganisms are not added and the digestion is performed in the bioreactor system while using microorganisms present in the separated lignocelluloses.

Digestion of the cellular matter to produce biogas includes digestion by hydrolysis-promoting, acid-forming microorganisms, and methanogenic microorganisms, as described hereinabove, and optionally other microorganisms. The acid-forming microorganisms form acetate, long-chain fatty acids, carbon dioxide, H₂, NH₂, and H₂S, as described hereinabove. The methanogenic microorganisms produce methane and carbon dioxide.

In the first step of the digestion process, polymeric substrates such as polysaccharides, proteins, and lipids are hydrolyzed into smaller subunits. In the second step, the hydrolyzed compounds are fermented to produce acetate, long-chain fatty acids, CO₂, H₂, NH₄ and H₂S. In a parallel step, proton-reducing acetogenic microorganisms (syntrophic organisms) degrade propionate, long-chain fatty acids, alcohols, amino acids, and aromatic compounds to H₂, and acetic acid. Degradation of these compounds with production of H₂ is sometimes harmful to the anaerobic digestion process unless the concentration of H₂ is maintained low by H₂-utilizing methanogenic microorganisms. Thus, the third step involves two different groups of methanogens, the hydrogenotrophic methanogens that use the H₂ produced by other microbes to reduce CO₂ to CH₄, and the acetotrophic methanogens that metabolize acetic acid to form CO₂ and CH₄.

In some embodiments, the separated lignocellulose may progress through a bioreactor system which comprises zones of optional disruption (e.g., by sonication), acid formation, and methane formation as the separated lignocellulose is mixed, folded and advanced through the bioreactor. The zones as described herein may be overlapping or be discrete zones. The zones may be present in one bioreactor or alternatively, the zones may be present in more than one bioreactor operatively linked sequentially to form a bioreactor system. For example, each zone may be present in a separate bioreactor.

In an exemplary bioreactor system, a first zone of a bioreactor or bioreactor system as described herein is a hydrolysis zone, where the lignocellulose material is disintegrated, disrupted and broken down into simpler, smaller compounds. In a second zone, the acid zone, microbial digestion occurs wherein acid-forming microorganisms, including acetogenic microorganisms begin to breakdown the polymers and smaller subunits as soon as they are formed. As more, smaller subunits become available, the number of acid-forming microorganisms present in the cellular matter multiply so that the acid zone becomes conditioned with a higher density of acid-forming microorganisms in a section of the acid zone toward the second end of the bioreactor. In a third zone of the bioreactor, the methane zone, another population of microorganisms known generally as methanogenic microorganisms further degrade products formed in the acid zone to produce biogas, including methane and/or carbon dioxide. It is to be noted that while exemplary 3 zones are described herein for a bioreactor or a bioreactor system, more or less zones or bioreactors are also contemplated.

In some embodiments, exogenous microorganisms, such as bacteria, may be supplied to any of the zones encompassing microbial degradation. The amount of exogenous microbes added will depend on the type and concentration of the material being digested and the amount of resulting product desired. The amount of exogenous microorganisms added should be sufficient to enhance production of the desired product in comparison to the production of the product without the addition of exogenous microorganisms.

In some embodiments, the microorganisms present in the bioreactor may be continually used in the respective zones as long as new lignocellulosic material is added to provide new substrate on which the microorganisms may continue to grow. Alternatively, the lignocellulosic material may be digested in a batch-wise manner wherein an amount of lignocellulose is supplied to the bioreactor, subjected to microbial digestion, and removed from the bioreactor before new material is added.

In some of any of the embodiments described herein, sonication may be applied to the lignocellulose material in any zone.

Herein throughout, the terms “sonicate”, “sonicated”, “sonicating” and “sonication” refer to irradiation by a sound wave, for example, a sound wave or ultrasound wave in a range of from 1 kHz to 2 MHz, optionally from 1 kHz to 1 MHz.

In some of these embodiments, a sound or ultrasound irradiation is applied in the hydrolysis zone, for promoting disintegration and hydrolysis of the lignocelluloses. Alternatively or in addition, a sound or ultrasound irradiation is applied to the acid zone and/or the methane zone. The sonication is applied using a sound or ultrasound generating system as described herein. In some embodiments, the frequency range for the sound or ultrasound irradiation supplied to the hydrolysis zone, as described herein, is in the frequency range from about 1 kHz to about 10 kHz. The frequency range for the sound or ultrasound irradiation supplied to the acid zone and the methane zone, and any additional zone, is in the frequency range from about 1 kHz to about 2,000 kHz.

In some embodiments, the method further comprises mixing, folding, and advancing the separated lignocellulose from the first end of the bioreactor or bioreactor system to the second end using a rotating member operatively connected to the bioreactor or bioreactor system.

Mixing may be intermittent or continuous. In addition, the number of rotations per minute (rpm) of the rotating member may vary and are adjustable depending on the process requirements.

In some embodiments, the method further comprises removing the biogas formed in the methane zone and forming a vacuum in the bioreactor by removing the biogas. The biogas may be removed using a pump to draw off the gas formed. Removing the biogas may be performed by any technique commonly known in the art for removing gas from a reactor and maintaining a vacuum. The method further comprises intermittently removing accumulation of residues from the bioreactor or bioreactor system.

In some embodiments, the method may further comprise providing a heating means for at least a portion of the bioreactor or bioreactor system. The heating means may be provided on the exterior of the bioreactor for the portion of the reactor in which the microbial digestion occurs, for example the acid zone and the methane zone. Providing a heating means allows the methanogenic degradation to occur at thermophilic temperatures. The term thermophilic as used herein describes temperatures in the range of about 50° C. to about 60° C.

In some of the any of the embodiments described herein, the microbial digestion as described herein in any of the respective embodiments is performed in a bioreactor or a bioreactor system configured for effecting the microbial digestion as described herein. The bioreactor or bioreactor system is having an inlet at a first end and an outlet at a second end. The bioreactor or bioreactor system may optionally further comprise a sound or ultrasound generating system operatively connected to the bioreactor to supply sound or ultrasound waves to at least one zone within the bioreactor. The sound or ultrasound generating system further comprises a power supply, a wave-form generator, a transducer, and a contact plate. The sound or ultrasound generating system supplies the sound or ultrasound irradiation to the at least one zone as described above.

The bioreactor or bioreactor system, in some embodiments, further comprises at least one rotating member to mix, fold, and advance the digested material in the bioreactor or bioreactor system from the first end to the second end of the bioreactor or bioreactor system. The at least one rotating member rotates about a shaft operatively connected to the bioreactor(s). The shaft may be driven by an electric motor. The electric motor provides a variable frequency drive, although a constant frequency drive may also be used to drive the shaft.

The bioreactor or bioreactor system may further comprise a means for supplying microorganisms as described herein.

The bioreactor or bioreactor system may further comprise a gas exhaust valve operatively connected to the bioreactor or bioreactor system. The valve may be connected to a pump to draw off the biogas formed in the bioreactor or bioreactor system, thereby creating a vacuum in a headspace formed in the bioreactor or bioreactor system. The bioreactor or bioreactor system may further comprise a heating means for supplying heat to a portion of the bioreactor. In an embodiment of the present invention, the second end of the bioreactor is elevated with respect to the first end. The degree of elevation is adjustable.

In some of the embodiments in which the system comprises a plurality of bioreactors, a first bioreactor is operatively connected to at least one more bioreactor(s). In some embodiments, the first bioreactor comprises a first sound or ultrasound generating system that sonicates at a frequency range of about 1 kHz to about 10 kHz, as described herein. The first bioreactor may further comprise at least one rotating member and a process controller.

The connection between the first bioreactor and the at least one more bioreactor may be a physical connection, or alternatively, the connection may comprise the transfer of material from the first bioreactor to the at least one more bioreactor without physical contact between the bioreactors. The at least one more bioreactor may comprise at least one more sound or ultrasound generating system for sonicating cellular matter to at a frequency range of about 1 kHz to about 2,000 kHz.

Ethanol Production:

In some of any of the embodiments described herein, a method as described herein is used for producing ethanol (also referred to herein as in the art as bioethanol), and is effected by fermentation using any of the enzymes and yeasts known in the art to effect ethanol or other alcohol production from soluble carbohydrates (e.g., soluble sugars such as glucose or xylose).

An exemplary fermentation process typically starts by breaking down the lignocellulose into complex sugars, typically by means of acidic solution and/or by microbial enzymes. This step is also referred to herein and in the art as pre-treatment. The method starts with pre-treating the separated lignocelluloses, e.g., by acid hydrolysis under adequate conditions of pressure and temperature, in order to break the lignin seal and disrupt the crystalline structure of cellulose. This causes solubilization of the hemicellulose and cellulose fractions. The cellulose and hemicellulose can then be hydrolyzed enzymatically, e.g., by cellulase enzymes (cellulolytic enzymes), to convert the carbohydrate polymers into fermentable sugars which, using a fermenting organism, e.g. a yeast, may be fermented into a desired fermentation product, such as ethanol. Optionally the fermentation product may be recovered, e.g., by distillation.

A glucoamylase is an exemplary enzyme added to break the complex sugars down into simple sugars. Typically, the yeast species Saccharomyces cerevisiae, and optionally genetically engineered mutants thereof, is used to convert carbohydrates to carbon dioxide and ethanol. Other microorganisms which are usable in fermentation to produce ethanol include, but are not limited to, Zymomonas mobilis, and Schizosaccharomyces. Further microorganisms are listed hereinafter.

The following describes an exemplary method for processing lignocellulosic material (a separated lignocelluloses as described herein) by fermentation so as to produce ethanol.

Methods for pre-treating lignocellulose-containing material are well known in the art. The pre-treated lignocellulose degradation products include lignin degradation products, cellulose degradation products and hemicellulose degradation products. The pre-treated lignin degradation products may be phenolics in nature.

The hemicellulose degradation products include furans from sugars (such as hexoses and/or pentoses), including xylose, mannose, galactose, rhamanose, and arabinose. Examples of hemicelluloses include xylan, galactoglucomannan, arabinogalactan, arabinoglucuronoxylan, glucuronoxylan, and derivatives and combinations thereof.

The lignocellulose-containing material may be pre-treated in any suitable way. Pre-treatment may be carried out before and/or during hydrolysis and/or fermentation. In some embodiments the pre-treated material is hydrolyzed, preferably enzymatically, before and/or during fermentation.

According to some embodiments, the pre-treatment may be a conventional pre-treatment step using techniques well known in the art. Examples of suitable pre-treatments are described hereinafter.

The separated lignocellulose material may be chemically, mechanically and/or biologically pre-treated before hydrolysis and/or fermentation. Preferably, chemical, mechanical and/or biological pre-treatment is carried out prior to the hydrolysis and/or fermentation. Alternatively, the chemical, mechanical and/or biological pre-treatment may be carried out simultaneously with hydrolysis, such as simultaneously with addition of one or more cellulase enzymes (cellulolytic enzymes), or other enzyme activities mentioned below, to release, e.g., fermentable sugars, such as glucose and/or maltose.

The term “chemical pre-treatment” refers to any chemical pre-treatment which promotes the separation and/or release of cellulose, hemicellulose and/or lignin. Examples of suitable chemical pre-treatments include treatment with; for example, dilute acid, lime, alkaline, organic solvent, ammonia, sulfur dioxide, carbon dioxide. Further, wet oxidation and pH-controlled hydrothermolysis are also considered chemical pre-treatment.

In some embodiments the chemical pre-treatment is acid treatment, for example, a continuous dilute and/or mild acid treatment, such as, treatment with sulfuric acid, or another organic acid, such as acetic acid, citric acid, tartaric acid, succinic acid, hydrogen chloride or mixtures thereof. Other acids may also be used. Mild acid treatment means that the treatment pH is in a range of from 1 to 5, preferably from 1 to 3. An exemplary acid concentration is in a range of from 0.1 to 2.0 weight percents acid, preferably sulphuric acid. The acid may be contacted with the separated lignocelluloses and the mixture may be held at a temperature in the range of 160-220° C., for a time period ranging from minutes to seconds, e.g., 1-60 minutes. Alternatively, alkaline H₂O₂, ozone, organic solvents such as Lewis acids, FeCl₃, Al₂SO₄ in aqueous alcohols, glycerol, dioxane, phenol, or ethylene glycol are used to disrupt cellulose structure and promote hydrolysis. Alkaline chemical pre-treatment with base, e.g., NaOH, Na₂CO₃ and/or ammonia or the like, can also be used.

Wet oxidation techniques involve use of oxidizing agents, such as: sulphite based oxidizing agents or the like. Examples of solvent pre-treatments include treatment with DMSO (Dimethyl Sulfoxide) or the like.

Chemical pre-treatment is generally carried out for 1 to 60 minutes, such as from 5 to 30 minutes, but may be carried out for shorter or longer periods of time dependent on the material to be pre-treated.

The term “mechanical pre-treatment” refers to any mechanical (or physical) treatment which promotes the separation and/or release of cellulose, hemicellulose and/or lignin from lignocellulose-containing material. For example, mechanical pre-treatment includes various types of milling, irradiation, steaming/steam explosion, and hydrothermolysis.

In some embodiments, mechanical pre-treatment includes sonication, as described herein.

Alternatively or in addition, mechanical pre-treatment includes comminution (mechanical reduction of the size). Comminution includes dry milling, wet milling and vibratory ball milling. Mechanical pre-treatment may involve high pressure and/or high temperature (steam explosion).

In some embodiments both chemical and mechanical pre-treatments are applied. For example, the pre-treatment may involve dilute or mild acid treatment and sonication, high temperature and/or pressure treatment. The chemical and mechanical pre-treatment may be carried out sequentially or simultaneously, as desired.

As used herein the term “biological pre-treatment” refers to any biological pre-treatment which promotes the separation and/or release of cellulose, hemicellulose, and/or lignin from the lignocellulose-containing material. Biological pre-treatment techniques can involve applying lignin-solubilizing microorganisms.

In some embodiments the pre-treated separated lignocellulose may be washed.

It is to be noted that any of the pre-treatment methods described herein are optional since it is shown herein that some pre-treatment of the lignocelluloses can already be effected when a waste material is subjected to separation according to specific gravity, when a salt solution at a salt concentration higher than about 10 weight percents is used.

It is to be further noted that when chemical pre-treatment is applied, for example, treatment with an acidic solution, as described herein, such a treatment can be effected prior to obtaining a fraction of a separated lignocelluloses (for example, by treating the waste material prior to separation, and/or a fraction, e.g., first fraction, prior to separation in a second aqueous liquid), and/or subsequent to obtaining the separated lignocellulose. With reference to FIG. 1, treatment with an acidic solution can be effected following separation in water 152, and/or before fermentation 162 or microbial digestion 163.

The hydrolysis of pre-treated lignocelluloses can be effected before and/or simultaneously with fermentation of the pre-treated lignocellulose-containing material. Hydrolysis may be carried out as a fed batch process in which the pre-treated lignocellulose material (substrate) is fed gradually to an, e.g., enzyme containing hydrolysis solution, or a bioreactor containing such a solution.

In some embodiments, hydrolysis is carried out enzymatically. In some embodiments, the pre-treated lignocellulose material may be hydrolyzed by one or more hydrolases (class EC 3 according to Enzyme Nomenclature), preferably one or more carbohydrases such as, but not limited to, cellulase, hemicellulase, amylase, such as alpha-amylase, protease, carbohydrate-generating enzyme, such as glucoamylase, esterase, such as lipase.

The enzyme(s) used for hydrolysis is (are) capable of directly or indirectly converting carbohydrate polymers into fermentable sugars which can be fermented into a desired fermentation product, such as ethanol.

In some embodiments, the carbohydrase has cellulase enzyme activity.

In a preferred embodiment the pre-treated lignocellulose-containing material is hydrolyzed using a hemicellulase, preferably a xylanase, esterase, cellobiase, or combination thereof.

Hydrolysis may also be carried out in the presence of a combination of hemicellulases and/or cellulases.

Enzymatic treatments may be carried out in a suitable aqueous environment under conditions which can readily be determined by one skilled in the art.

Suitable hydrolysis time, temperature and pH conditions can readily be determined by one skilled in the art. In exemplary embodiments, hydrolysis is carried out at a temperature of from 25 to 70° C., or from 40 and 60° C. The hydrolysis can be performed at a pH in the range from 3 to 8, or from 4 to 6.

The hydrolysis can be effected during a time period that ranges from 12 to 96 hours, or from 16 to 72 hours, or from 24 to 48 hours.

The pre-treated (and hydrolyzed) lignocellulose material is fermented by at least one fermenting organism capable of fermenting fermentable sugars, such as glucose, xylose, mannose, and galactose directly or indirectly into a desired fermentation product.

The term “fermenting organism” refers to any organism, including bacterial and fungal organisms, suitable for producing a desired fermentation product. Examples of fermenting organisms include, but are not limited to, fungal organisms, such as yeast. Preferred yeast includes strains of the genus Saccharomyces, in particular a strain of Saccharomyces cerevisiae or Saccharomyces uvarum; a strain of Pichia, in particular Pichia stipitis or Pichia pastoris; a strain of the genus Candida, in particular a strain of Candida utilis, Candida arabinofermentans, Candida diddensii, or Candida boidinii.

Other contemplated yeast includes strains of Hansenula, in particular Hansenula polymorpha or Hansenula anomala; strains of Kluyveromyces, in particular Kluyveromyces marxianus or Kluyveromyces fagilis, and strains of Schizosaccharomyces, in particular Schizosaccharomyces pombe.

Exemplary bacterial fermenting organisms include strains of Escherichia, in particular Escherichia coli, strains of Zymomonas, in particular Zymomonas mobilis, strains of Zymobacter in particular Zymobacter palmae, strains of Klebsiella, in particular Klebsiella oxytoca, strains of Leuconostoc, in particular Leuconostoc mesenteroides, strains of Clostridium, in particular Clostridium butyricum, strains of Enterobacter in particular Enterobacter aerogenes and strains of Thermoanaerobacter, in particular Thermoanaerobacter BG1L1 and Thermoanaerobacter ethanolicus

The pH during the fermentation may be in a range of from 5.5 to 9.0, or from 5.7 to 8.0, or from 5.8 to 7.0, or from pH 5.9 to 6.5. The pH may be adjusted using any suitable compound. In some embodiments, the pH is adjusted using NaOH.

In some embodiments, the dry solids concentration during the fermentation is in a range of from about 20% to about 35% by weight.

The fermentation is typically carried out during a time period that ranges from 8 to 96 hours, or from 12 to 72 hours, or from 24 to 48 hours.

In some embodiments the fermentation is carried out at a temperature of from 20 to 40° C., or from 26 to 34° C., or at about 32° C.

According to some embodiments, the hydrolysis and fermentation may be carried out simultaneously or sequentially. When carried out simultaneously, both the hydrolysis-catalyzing enzyme and the fermenting organism are added to a solution containing the lignocellulose, optionally within a bioreactor. In some of these embodiments, the combined/simultaneous hydrolysis and fermentation are carried out at conditions (e.g., temperature and/or pH) suitable for the fermenting organism(s) in question. A temperature program comprising at least two holding stages at different temperatures may be optionally applied.

In some embodiments hydrolysis and fermentation are carried out as hybrid hydrolysis and fermentation, which typically begins with a separate partial hydrolysis step and ends with a simultaneous hydrolysis and fermentation step. The separate partial hydrolysis step is an enzymatic cellulose saccharification step typically carried out at conditions (e.g., at higher temperatures) suitable for the hydrolyzing enzyme(s) in question. The subsequent simultaneous hydrolysis and fermentation step is typically carried out at conditions suitable for the fermenting organism(s) (often at lower temperatures than the separate hydrolysis step).

In some embodiments, the hydrolysis and fermentation are carried out separately such that the hydrolysis is completed before initiation of fermentation. Such a process can be carried out in the same bioreactor or in different bioreactors, such hydrolysis is carried out in a first bioreactor and once complete, the material is transferred to a second bioreactor, which includes the fermenting organism.

Subsequent to fermentation the fermentation product may be separated from the fermentation medium/broth. The medium/broth may be distilled to extract the fermentation product or the fermentation product may be extracted from the fermentation medium/broth by micro or membrane filtration techniques. Alternatively the fermentation product may be recovered by stripping. Recovery methods are well known in the art.

The System:

According to another aspect of embodiments of the invention there is provided a system for processing a waste material so as to form a biogas and/or ethanol. The system comprises at least one chamber configured for receiving a waste material, and containing an aqueous liquid selected such that at least a portion of the waste material sinks upon contact with the aqueous liquid (e.g., an aqueous liquid according to any of the respective embodiments described herein).

Herein, the term “separator” refers to a chamber containing a liquid and configured as described hereinabove, thereby being capable of effecting a cycle of contacting an inputted material (e.g., waste material or fraction thereof) with an aqueous liquid and separation of oil (e.g., as described herein).

In some embodiments of any of the embodiments pertaining to a system described herein, the system is configured for removing at least a portion of low-density solid materials from the aqueous liquid in the chamber, to thereby obtain a first fraction of solid materials. In some embodiments, the system further comprises an apparatus configured for removing at least a portion of a liquid from the first fraction of solid materials (e.g., by compression and/or drainage, as described herein), to thereby obtain a liquid fraction. In some embodiments the apparatus for removing at least a portion of a liquid from the first fraction of solid materials comprises a screw press.

As used herein the term “about” refers to ±10%.

The terms “comprises”, “comprising”, “includes”, “including”, “having” and their conjugates mean “including but not limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integer numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### General Procedure for Separating Lignocellulose from Waste Material

A general procedure 100 for separating waste material according to some embodiments of the present invention is shown in FIG. 1.

Waste material 110 is provided, optionally “wet” waste material, i.e., waste material which has not been subjected to drying, and optionally wet substantially unsorted waste material (SUW). The waste material is preferably domestic waste material, e.g., collected from private households. Optionally, the waste material has been subjected to preliminary processing procedures (e.g., at a waste disposal facility), such as crushing (e.g., by a hammer mill), and/or removal of magnetic materials.

Waste material 110 is subjected to separation in salt solution 115 (by contacting the waste material 110 with a salt solution), resulting in separation of waste material 110 into a first fraction of low-density materials 130 and a second fraction of high-density materials 120. Separation in salt solution 115 may optionally utilizes a salt solution (e.g., sodium chloride solution) having a specific gravity of at least 1.05, optionally at least 1.07, optionally at least 1.10, optionally at least 1.15 and optionally at least 1.20, as described herein. Optionally, separation in salt solution 115 further comprises obtaining oil-rich liquid 140 (comprising of or consisting of oil) from a surface of the salt solution, for example, by skimming. First fraction 130 is optionally subjected to shredding 132, resulting in shredded low-density materials.

First fraction 130, which is wet, is then subjected to liquid removal 134, to thereby obtain partially wet first fraction 150 and liquids 155. Liquid removal 134 optionally comprises compression (e.g., by screw press) and/or draining (driven by gravity and/or compression).

Partially wet first fraction 150 is subjected to separation in water 152 (by contacting material of fraction 150 with water), resulting in separation of material of fraction 150 into a third fraction of low-density materials 170 (optionally low-density polymeric material, e.g., polyolefins) and a fourth fraction of intermediate-density materials 160 (comprising separated lignocellulose as described herein). Separation in water 152 may optionally utilizes an aqueous liquid (e.g., pure water or dilute aqueous solution) having a specific gravity of no more than 1.03, optionally no more than 1.02, optionally no more than 1.01, and optionally no more than 1.00, as described herein. Optionally, separation in water 152 further comprises obtaining oil-rich liquid 172 (comprising of or consisting of oil), for example, by skimming a surface of the water.

Separation in salt solution 115 and separation in water 152 are each optionally performed using a separator as described herein, containing the appropriate liquids.

Fourth fraction 160 containing lignocellulose-enriched material is then introduced into a bioreactor of a bioreactor system to produce a biogas as described herein in any of the respective embodiments and/or into a bioreactor or a bioreactor system to produce ethanol as described herein in any of the respective embodiments.

Fourth fraction 160 is optionally subjected to fermentation or microbial digestion process 162, which is adapted to result in a fermentation product such as ethanol 164 (by fermentation) and/or biogas 166 (by microbial digestion). Material from fourth fraction 160 which is not converted to a fermentation product such as ethanol 164 and/or biogas 166 remains as organic residue 168. Fourth fraction 160 and organic residue 168 may each be optionally used to form a compost.

Third fraction 170 is optionally processed by heating a feedstock comprising thirst fraction 170, to produce a relatively homogeneous processed polymeric material. The feedstock may optionally comprise additional materials, including fourth fraction 160 and/or organic residue 168. Third fraction 170, fourth fraction 160 and/or organic residue 168 may be included in pre-determined proportions in the feedstock, the proportions depending on the desired properties of the processed material and/or the relative cost effectiveness of different combinations of third fraction 170, fourth fraction 160 and organic residue 168.

FIG. 2 presents a schematic illustration of a system for performing the general procedure herein described.

System 200 comprises a separator 210, and optionally and preferably further comprises a second separator 250, separators 210 and 250 each being for separating material into at least two fractions, according to specific gravity.

In some embodiments, separator 210 separates waste material into a first fraction comprising a low-density material which does not sink in an aqueous liquid in separator 210 (optionally a salt solution) and a second fraction comprising a high-density material which sinks in the liquid.

In some embodiments, system 200 comprises a second separator 250, which receives material from the first fraction and/or second fraction from separator 210, optionally via conduit 232. System 200 is optionally configured such that material from either the first fraction or the second fraction may be received by separator 250, in a controllable and reversible manner. The material may be received after passing through shredder 230 (as depicted in FIG. 2) which is optionally connected to separator 210 by conduit 214, although passage of material from separator 210 to separator 250 without passing through shredder 230 is also contemplated.

In some embodiments, separator 250 separates material received directly or indirectly from separator 210 into a fraction comprising a low-density material which does not sink in an aqueous liquid in separator 250 (optionally water) and a fraction comprising a high-density material which sinks in the liquid. In some embodiments, separator 250 separates material from a first fraction described herein into a third fraction comprising a low-density material which does not sink in an aqueous liquid in separator 250 (optionally water) and a fourth fraction comprising an intermediate-density material which sinks in the liquid and comprises separated lignocellulose. Additionally or alternatively, separator 250 separates material from a second fraction described herein into a fifth fraction comprising a high-density material which sinks in an aqueous liquid in separator 250 (optionally a salt solution) and a fourth fraction comprising an intermediate-density material which does not sink in the liquid and comprises separated lignocellulose.

A bioreactor or bioreactor system 260 for microbial digestion or fermentation (as described herein) receives separated lignocellulose from separator 250 (as depicted in FIG. 2) and/or separator 210 (not shown), optionally via conduit 252. Bioreactor or bioreactor system 260 is optionally configured for producing a biogas and/or ethanol from separated lignocellulose by microbial digestion or fermentation.

In some embodiments, system 200 further comprises inlets and outlets in some or all of its components, for allowing communication between the components.

In some embodiments, system 200 further comprises collector units for collecting the separated materials (e.g., in any of the fractions described herein) or the processed materials (e.g., biogas and/or ethanol) as described herein.

### Example 2

### Effect of Hypertonic Solution on Biomass in Waste Material

6 grams of fresh organic waste (carrot, cucumber, banana peels) was placed in samples of 60 ml fresh water or 60 ml of salt water with about 20 weight percents salt, and incubated at room temperature for 3 hours. Filtrates of each sample were then analyzed by solid-state NMR spectroscopy, performed using a Chemagnetics™ Infinity console (300 MHz proton frequency) with a Chemagnetics™ triply resonant variable temperature probe.

FIGS. 3A and 3B present ¹³C spectra of the obtained filtrates, and show that the filtrate from the salt solution exhibited NMR signals in a range of from 60-100 ppm (FIG. 3A), typical of carbohydrates such as glucose and xylose, whereas no such signals were observed for the filtrate obtained from fresh water (FIG. 3B).

These results indicate that the use of hypertonic solutions to separate waste material breaks cell walls and facilitates release of carbohydrates.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of processing a waste material so as to form a biogas and/or ethanol, the method comprising:
subjecting the waste material to a separation according to specific gravity, to thereby obtain at least one fraction which is a separated lignocellulose; and
processing said separated lignocellulose, to thereby obtain the biogas and/or ethanol.

2. The method of claim 1, wherein processing said separated lignocellulose is performed so as to produce biogas, said processing comprising subjecting said separated lignocelluloses to a microbial digestion in the presence of an acetogenic microorganism and a methanogenic microorganism.

3. The method of claim 1, wherein processing said separated lignocellulose is performed so as to produce ethanol, said processing comprising subjecting said separated lignocelluloses to a fermentation in the presence of a fermenting organism.

4. The method of claim 1, further comprising, prior to or concomitant with said processing, pre-treating said separated lignocellulose so as to at least partially decompose the lignocellulose into lignin, hemicelluloses and cellulose.

5. The method of claim 1, wherein said separation according to specific gravity comprises contacting the waste material with an aqueous liquid selected such that a portion of the waste material sinks and another portion does not sink, thereby separating waste material into a first fraction comprising a low density material and a second fraction comprising a high-density material.

6. The method of claim 5, wherein said first fraction comprises said separated lignocellulose.

7. The method of claim 5, wherein said separation process comprises contacting the waste material with a first aqueous liquid selected such that a portion of said waste material sinks, thereby obtaining said second fraction comprising said high-density material and said first fraction comprising said low-density material, and further contacting at least one of said first fraction and said second fraction with a second aqueous liquid selected such that a portion of said fraction sinks, thereby obtaining a third fraction comprising a low-density material which does not sink in either of said aqueous liquids, a fourth fraction comprising an intermediate-density material which sinks in one of said aqueous liquids, and a fifth fraction comprising a high-density material which sinks in both of said aqueous liquids.

8. The method of claim 7, wherein a specific gravity of one of said first aqueous liquid and said second aqueous liquid is at least 1.05, and a specific gravity of the other of said first aqueous liquid and said second aqueous liquid is no more than 1.01.

9. The method of claim 7, wherein said intermediate-density material comprises said separated lignocellulose.

10. A system for processing a waste material so as to form a biogas and/or ethanol, the system comprising:
at least one separator configured for separating materials in the waste material according to specific gravity so as to obtain at least two fractions, said fractions comprising at least a first fraction which comprises a low density material and at least a second fraction which comprises a high-density material, said separator containing an aqueous liquid selected such that a portion of said waste material sinks and another portion does not sink upon contact with said aqueous liquid, thereby obtaining said first fraction and said second fraction; and
a bioreactor or a bioreactor system configured for processing said separated lignocellulose to thereby obtain the biogas and/or ethanol.

11. The system of claim 10, wherein said bioreactor or a bioreactor system is configured for processing said separated lignocellulose so as to produce the biogas, said processing comprising subjecting said separated lignocellulose to a microbial digestion in the presence of an acetogenic microorganism and a methanogenic microorganism.

12. The system of claim 10, wherein said bioreactor or a bioreactor system is configured for processing said separated lignocellulose so as to produce ethanol, said processing comprising subjecting said separated lignocelluloses to a fermentation in the presence of a fermenting organism.

13. The system of claim 10, wherein said bioreactor or bioreactor system is in communication with at least one of said at least one separator, and is configured for processing at least a portion of said first fraction which comprises a low-density material.

14. The system of claim 10, wherein said at least one separator comprises a first separator containing a first aqueous liquid and a second separator containing a second aqueous liquid, said first separator and said second separator being in communication, and said second separator being configured for receiving at least one fraction from said first separator, and for separating said fraction received from said first separator according to specific gravity, said second aqueous liquid being selected such that a portion of said fraction received from said first separator sinks, thereby obtaining a third fraction comprising a low-density material which does not sink in either of said aqueous liquids, a fourth fraction comprising an intermediate-density material which sinks in one of said aqueous liquids, and a fifth fraction comprising a low-density material which sinks in both of said aqueous liquids.

15. The system of claim 14, wherein a specific gravity of one of said first aqueous liquid and said second aqueous liquid is at least 1.05, and a specific gravity of the other of said first aqueous liquid and said second aqueous liquid is no more than 1.01.

16. The system of claim 14, wherein said second separator is configured for obtaining a separated lignocellulose, said intermediate-density material comprising said lignocellulose.

17. The system of claim 14, wherein said bioreactor or bioreactor system is in communication with said second separator, sand is configured for processing at least a portion of said fourth fraction which comprises an intermediate-density material.
